(19) United States
(12) Patent Application Publication
(10) Pub. No.: US 2008/0102054 A1
(43) Pub. Date: May 1, 2008

(54) Compositions Containing Agm Cells And Methods Of Use Thereof

(76) Inventor: Denise L. Faustman, Boston, MA (US)

Correspondence Address: CLARK & ELBING LLP, 101 FEDERAL STREET, BOSTON, MA, 02110, US

(21) Appl. No.: 11/795,540

(22) PCT Filed: Jan. 18, 2006
(86) PCT No.: PCT/US06/01843

§ 371 Date: Jul. 18, 2007

Related U.S. Application Data
(60) Provisional application No. 60/644,748, filed on Jan. 18, 2005.

(51) Int. Cl. A61K 38/20; C12N 5/08; A61P 31/00; A61P 3/10; A61K 38/21; A61K 48/00; A61K 38/19

(52) U.S. Cl. 424/85.2; 424/93.7; 435/366; 424/85.4

(57) ABSTRACT

The invention features a therapeutic composition containing mammalian, preferably human, post-fetal, AGM cells in a pharmaceutically acceptable carrier. The compositions of the invention can be administered to treat patients suffering from autoimmune diseases, to treat patients in need of organ or cell regeneration, and to treat patients in need of immune, especially hematopoietic, reconstitution.

### BACKGROUND OF THE INVENTION

The spleen is an organ that filters blood and contributes to the immune response. Situated at the intersection of the immune and circulatory systems, the spleen is loosely divided into histological areas of red and white pulp. The red pulp possesses macrophages, whose normal role is to filter and remove from the blood senescent and dysfunctional red blood cells (RBCs), as well as antibody-coated bacteria or RBCs. The higher concentration of RBCs gives the red pulp its name. The white pulp, a lymphoid compartment, plays a pivotal role in immune surveillance and response. The white pulp releases antibodies to counter invading pathogens and releases platelets and neutrophils to counteract bleeding or infection. The spleen also has a storage function and houses up to one-third of the body's platelets.

Pluripotent stem cells, cells which are capable of developing into several different cell lineages of the body, are known to exist in the bone marrow of adult mammals. Pluripotent stem cells are also known to reside outside the bone marrow, e.g., in the umbilical cord blood and peripheral blood, as well as in several mature tissues, including muscle, brain, skin, liver, and mammary gland (Kuehnle and Goodell, Br. Med. J. 325: 372-6; 2002; Rosenthal, New Eng. J. Med. 349: 267-74; 2003). Stem cells, with their capacity for self-renewal and differentiation to a variety of specialized cells, are advantageous for replacing diseased or damaged tissue. The first advantage is that few cells would need to be harvested because their number could be readily expanded in vivo or ex vivo, given their potent proliferative capacity. The second advantage is that autologous stem cells (that is, stem cells from the patient's own body) could be used, thereby overcoming the need for long-term immunosuppressive therapy. The third advantage is that autologous (or heterologous) cells can be harvested from an adult, thereby avoiding the more ethically controversial destruction of an embryo.

Pluripotent stem cells for use in replacing diseased or damaged tissue have been identified thus far in bone marrow, peripheral blood, cord blood, liver, and pancreas of adult animals. Studies in both mice and in humans have shown that the introduction of MHC-matched bone marrow-derived stem cells into irradiated hosts results both in repopulation of the host bone marrow as well as rare examples of donor engraftment of host parenchymal organs, including the liver, brain, muscle, and heart, with scattered tissue-specific cells. Such engraftment, though, is typically neither robust nor durable. Furthermore, there has been disagreement on which cell types participate in the regeneration of tissue and to what extent the cells promote that regeneration. For example, some reports indicate that the effect of the adult stem cells, once transplanted, is not direct. Rather, the adult stem cells stimulate regeneration by endogenous cells, possibly by release of growth factors.

Given the uncertainty in the field, there exists a need for adult, pluripotent stem cells having the ability to consistently engraft and regenerate diseased or damaged tissues and organs. Desirably, the adult, pluripotent stem cells need little or no damaging pre-treatment (such as, for example, irradiation or chemical treatment), can be obtained from an endogenous source, and can be expanded, induced, or stimulated. Ideally, the regenerative potential of the adult, pluripotent stem cells would be applicable not only to tissue damage that results from autoimmune attack, but also to non-autoimmune induced damage.

### SUMMARY OF THE INVENTION

The invention provides methods for organ or tissue regeneration in a mammal (e.g., a human patient). Accordingly, in a first aspect, the invention features a method for increasing or maintaining the number of functional cells of a predetermined type in an organ or tissue of a mammal who has injured or damaged cells of the predetermined type, or is deficient in the predetermined type of cells, that includes administering to the mammal a composition containing pluripotent cells that lack cell surface expression of CD45 (i.e., CD45−). In another embodiment, the pluripotent cells express the Hox11 gene. In another embodiment, the pluripotent cells are aorta gonad mesoderm (AGM) cells and at least 10% of the cells of the composition are AGM cells. In other embodiments, at least 20, 30, 40, 50, 60, 70, 80, 90, 95, 99% of the cells of the composition are AGM cells. In a preferred embodiment, the AGM cells are obtained from spleen, tonsils, adenoids, thymus, peripheral blood, or cord blood. More preferably, the AGM cells are obtained from spleen, e.g., the capsule or pulp of the spleen. In another preferred embodiment, the AGM cells are human cells.

In another embodiment of the first aspect, the Hox11-expressing pluripotent cell is derived from a pluripotent or totipotent cell transfected with a Hox11 gene.

In another aspect, the invention features a method for increasing or maintaining the number of functional cells of a predetermined type in an organ or tissue of a mammal who has injured or damaged cells of the predetermined type, or is deficient in the predetermined type of cells, that includes administering to the mammal a composition containing pluripotent cells that are CD45(−), with the proviso that pluripotent cells are not bone marrow cells or muscle cells. Examples of CD45(−) pluripotent cells that are useful in the present invention include peripheral blood lymphocytes, cord blood cells, and splenocytes. Preferably, the pluripotent cells are splenocytes. In another embodiment, the pluripotent cells express the Hox11 gene. In yet another embodiment, the pluripotent cells are aorta gonad mesoderm (AGM) cells and at least 10% of the cells of the composition are AGM cells. In other embodiments, at least 20, 30, 40, 50, 60, 70, 80, 90, 95, 99% of the cells of the composition are AGM cells. In a preferred embodiment, the AGM cells are obtained from spleen, tonsils, adenoids, thymus, peripheral blood, or cord blood. More preferably, the AGM cells are obtained from spleen, e.g., the capsule or pulp of the spleen. In another preferred embodiment, the AGM cells are human cells.

The pluripotent cells used in the methods of the present invention can be semi-allogeneic or isogeneic. Any of the compositions of the invention can further include moieties (e.g., cells) that present MHC class I and peptide, where the MHC class I has at least one allele that matches an MHC class I allele expressed by the mammal to whom the composition is administered to and the peptide is one that is derived from endogeneous cells of the mammal.

Compositions or cell populations enriched in cells that do not express CD45 protein can obtained by providing mammalian peripheral blood or tissue, such as, for example, the spleen, tonsils, adenoids, and thymus, containing pluripotent cells; separating the pluripotent cells from the blood or tissue; further separating the pluripotent cells into a first cell population that predominantly expresses CD45 protein (i.e., one that is predominantly CD45(+)) on the cell surface and a second cell population that predominantly does not express CD45 protein (i.e., one that is predominantly CD45(−)) on the cell surface; and selecting the second cell population. A cell population that is predominantly CD45(−) is one that contains more cells that do not express this protein on the cell surface than those that do. The detection of CD45 expression can be achieved using any technique known in the art, e.g., immunofluorescence detection using fluorescence activated cell sorting (FACS). Desirably, at least 75%, more desirably, at least 90%, and most desirably, at least 95% of the second cell population is populated with cells that do not express CD45. CD45(−) cell populations are obtained by removing those cells expressing CD45 through the use of affinity chromatography or by cell sorting techniques.

In any of the methods of the invention, the organ or tissue is stimulated prior to administering the pluripotent cell composition (i.e., a cell composition containing AGM cells). Stimulation can include the use of agents that damage or otherwise prepare the organ or tissue for new cell growth. Stimulating agents include TNF-alpha, TNF-alpha agonists, or TNF-alpha inducing substances such as, for example, complete Freund's adjuvant (CFA), ISS-ODN, microbial cell wall components with LPS-like activity, cholera particles, E. coli heat labile enterotoxin, E. coli heat labile enterotoxin complexed with lecithin vesicles, ISCOMS-immune stimulating complexes, polyethylene glycol, poly(N-2-(hydroxypropyl)methacrylamide), synthetic oligonucleotides containing CpG or CpA motifs, monophosphoryl lipid A, MPL, Bacillus Clamette-Guerin, γ-interferon, Tissue Plasminogen Activator, LPS, Interleukin-1, Interleukin-2, UV light, a lymphotoxin, cachectin, a TNFR-1 agonist, a TNFR-2 agonist, an intracellular mediator of the TNF-alpha signaling pathway, a NFκB inducing substance, IRF-1, STAT1, a lymphokine, or the combination of TNF-alpha and an anti-TNFR-1 antibody. Preferably, the stimulating agent is TNF-alpha, BCG, gamma-interferon, or CFA. Stimulating agents can be administered admixed with the composition, separately but concurrently with the composition, or any time prior to, preferably 6-12 hours before, administration of the AGM cell-containing composition.

In another embodiment, the CD45(−) and/or Hox11-expressing pluripotent AGM cells contain one of more cell surface markers selected from the group consisting of: retinoic acid receptor, estrogen receptor, EGF receptor, CD49b, VLA2, CD41, LFA-1, ITB2, CD29, NTC3 receptor, plasminogen receptor, transferrin receptor, TGF receptor, TNF receptor (e.g., TNF receptor 1), PDGF receptor, thyroid growth hormone receptor, and integrin beta 5.

In yet another embodiment, Hox 11 expression in a pluripotent AGM cell population can be induced ex vivo, followed by administration of the Hox 11-pluripotent AGM cell-containing composition to the mammal. In the methods of the invention, the pluripotent AGM cell-containing composition can be administered one or more times. Typically, the compositions are added daily, twice weekly, or weekly, the frequency of administration dependent upon the treated subject's response to therapy (i.e., the successful regeneration of organs or tissue of the predetermined type).

In any of the methods of the present invention, the functional cells of a predetermined type that are the subject of tissue/organ regeneration can be those of the pancreas (both endocrine and exocrine), salivary gland, pituitary gland, kidney, heart, olfactory system, ear, bone, liver, brain (including cerebellum, metencephalon-mesencephalon, cranial nerve, pallio-subpallial boundary, cerebrum, forebrain, and brachial arch), peripheral nervous system, central nervous system, spinal cord, mammary gland, or testes (e.g., beta islet cells and acinar cells of the pancreas; neurosecretory neurons of the hypothalamus; acini cells of the salivary glands; somatotrophs, mammotrophs, corticotrophs, thyrotrophs, gonadotrophs, chromophobes, and pituicytes of the pituitary gland; podocytes, endothelial cells, and mesangial cells of the kidney; myocytes of the heart; olfactory epithelia and olfactory receptor cells of the olfactory gland; hair cells of the organ of Corti, columnar cells, inner and outer pillar cells, inner and outer phalangeal cells, cells of Hensen, border cells, and type I and type II hair cells of the ear, osteoclasts, osteoblasts, and osteocytes of the bone; hepatocytes of the liver; neurons, glial cells, and neuroglia of the brain; epithelial and myoepithelial cells of the mammary gland; and Leydig cells of the testes).

In another aspect, the invention features a method for increasing or maintaining the number of functional cells of a predetermined type in an organ or tissue of a mammal who has injured or damaged cells of the predetermined type, or is deficient in the predetermined type of cells, that includes administering to the mammal a composition that contains pluripotent cells resulting from transfection of a pluripotent or totipotent cell, preferably a semi-allogeneic or isogeneic pluripotent cell, with a Hox 11 gene, preferably a human Hox 11 gene. Preferably, transfection of the pluripotent cell results in the expression of the Hox11 gene. Most preferably, the transfected cells are CD45(−). In one embodiment, the pluripotent cells are splenocytes or are obtained from cord blood. In another embodiment, the transfected cell is capable of differentiating into a pancreatic cell, a spleen cell, a liver cell, a kidney cell, a nerve cell, or a bone cell, most preferably a pancreatic cell.

In another aspect, the invention features a method for increasing or maintaining the number of functional cells of a predetermined type in an organ or tissue of a mammal who has injured or damaged cells of the predetermined type, or is deficient in the predetermined type of cells, that includes administering to the mammal an agent that induces and/or stimulates CD45(−) and/or Hox11-expressing pluripotent cells. In one embodiment, the pluripotent cells are not bone marrow cells. In another embodiment, the method promotes regeneration of an organ or a tissue from an organ by increasing or maintaining the number of functional cells of a predetermined type in the organ or tissue. In another embodiment, the organ or tissue is selected from pancreas, hypothalamus, salivary gland, pituitary gland, kidney, heart, olfactory gland, ear, bone, liver, brain, peripheral nervous system, central nervous system, spinal cord, mammary gland, and testes.

In an embodiment, suitable agents for inducing and/or stimulating CD45(−) and/or Hox11-expressing pluripotent cells are, or are those that induce or stimulate, cytokines, chemokines, or growth factors, which in turn induce or stimulate CD45(−) and/or Hox11-expressing pluripotent cells. Examples of these agents can be selected from the group consisting of epidermal growth factor (EGF), platelet-derived growth factor (PDGF), fibroblast growth factors (FGFs), transforming growth factor-beta (TGF-β), transforming growth factor-alpha (TGF-α), vascular endothelial growth factor (VEGF), erythropoietin (Epo), insulin-like growth factor-I (IGF-I), insulin-like growth factor-II (IGF-II), interleukins, tumor necrosis factor-alpha (TNF-α), tumor necrosis factor-beta (TNF-β), gamma-interferon (INF-γ), stromal cell-derived factor-1 (SDF-1), and colony stimulating factors (CSFs). In another embodiment, the agent is a gene therapy vector comprising a Hox 11 gene operably linked to a promoter where the vector induces the expression Hox 11 in the pluripotent cells.

In another embodiment, CD45(−) and/or Hox11-expressing pluripotent cells are quantitated before and after administration of the stimulating/inducing agent to the mammal. Quantitation can be aided by detecting a first marker, preferably a marker that is the result of Hox 11 gene expression in the Hox 11-expressing cells, or one selected from the group consisting of: retinoic acid receptor, estrogen receptor, EGF receptor, CD49b, VLA2, CD41, LFA-1, ITB2, CD29, NTC3 receptor, plasminogen receptor, transferrin receptor, TGF receptor, TNF receptor (e.g., TNF receptor 1), PDGF receptor, thyroid growth hormone receptor, and integrin beta 5. Quantitation can be further aided by detecting a second marker expressed by a control cell population or a second pluripotent cell population that is not present on the CD45(−) and/or Hox11-expressing pluripotent cells. The method used to detect the first marker can include the use of an antibody specific for the marker, preferably with a binding constant for the marker of 1.0 μM or less. The relative increase or decrease of CD45(−) and/or Hox11-expressing cells can be assessed by comparing the ratios of first marker to second marker both before and after administration of the composition. If it is determined that administration of the CD45(−) and/or Hox11-expressing cell stimulating/inducing agent does not result in an increase of CD45(−) and/or Hox11-expressing pluripotent cells, then an additional amount of the same stimulating/inducing agent, or a different stimulating/inducing agent, can be administered to the mammal.

In another aspect the invention features a method for increasing or maintaining the number of functional cells of a predetermined type in an organ or tissue of a mammal who has injured or damaged cells of the predetermined type, or is deficient in the predetermined type of cells, that includes administering to the mammal an agent that selectively inhibits (e.g. via induction of senescence), removes, or kills cell populations (such as, for example, T-lymphocytes) that interfere or prevent the trafficking of, differentiation of, or growth of pluripotent cells. The pluripotent cells can be isogeneic or semi-allogeneic. Preferably, these cells express Hox-11 and/or are CD45(−). Repeat administration of the agent, or the administration of different agents, can then be affected as needed during therapy. In one example, the levels of T-lymphocytes with an increased sensitivity to apoptosis (e.g., those deficient in the expression of CD180) can be assessed by obtaining a blood sample from the patient and quantitating the sensitive cells by techniques known to those skilled in the art, such as, for example, by FACS analysis. The agent or agents can then be added as required to reduce or eliminate the T-lymphocytes that are apoptotically sensitive. In another embodiment, the method promotes regeneration of an organ or a tissue from an organ by increasing or maintaining the number of functional cells of a predetermined type in the organ or tissue. In another embodiment, the method treats or ameliorates a disease or disorder, e.g., an autoimmune disease or disorder, in the mammal. In another embodiment, the organ or tissue is selected from pancreas, hypothalamus, salivary gland, pituitary gland, kidney, heart, olfactory gland, ear, bone, liver, brain, peripheral nervous system, central nervous system, spinal cord, mammary gland, and testes.

Any of the methods of the present invention can further include the inducement of damage to, or stimulation of, organ or tissue cells of a predetermined type prior to administering the pluripotent cell composition. Methods of the present invention can also include administering to the mammal, before, during, or after the administration of a CD45(−) and/or Hox11-expressing pluripotent cell composition or an agent that stimulates or induces CD45(−) and/or Hox11-expressing pluripotent cells, the administration of an agent that can selectively inhibit, remove, or kill cell populations that interfere or prevent the trafficking, differentiation, or growth of pluripotent cells. As before, repeat administration of the agent, or the administration of different agents, can then be affected as needed during therapy.

Agents that can selectively inhibit, remove, or kill cell populations (e.g., T-lymphocytes) that interfere or prevent the trafficking, differentiation, or growth of pluripotent cells include TNF-alpha, TNF-alpha agonists, or TNF-alpha inducing substances such as, for example, complete Freund's adjuvant (CFA), ISS-ODN, microbial cell wall components with LPS-like activity, cholera particles, E. coli heat labile enterotoxin, E. coli heat labile enterotoxin complexed with lecithin vesicles, ISCOMS-immune stimulating complexes, polyethylene glycol, poly(N-2-(hydroxypropyl)methacrylamide), synthetic oligonucleotides containing CpG or CpA motifs, monophosphoryl lipid A, Bacillus Clamette-Guerin, γ-interferon, Tissue Plasminogen Activator, LPS, Interleukin-1, Interleukin-2, UV light, a lymphotoxin, cachectin, a TNFR-1 agonist, a TNFR-2 agonist, an intracellular mediator of the TNF-alpha signaling pathway, a NFκB inducing substance, IRF-1, STAT1, a lymphokine, or the combination of TNF-alpha and an anti-TNFR-1 antibody. Preferably, the agent is TNF-alpha, CFA, gamma-interferon, or BCG.

Other examples of compounds that induce T-lymphopenia include compounds that bind or activate one or more members of the TNF receptor superfamily (e.g., TNF receptor 1 or 2, Trail-R1, Trail-R2, Trail-R3, Trail-R4, OPG, Rank<, Fn14, DR6, Hvem, LtbetaR, DcR3, Tramp, Fas, CD40, CD30, CD27, 4-1BB, OX40, Gitr, Ngfr, BCMA, Taxi, Baff-r, EDAR, Xedar, Troy, Relt, or CD95L). Therapeutic agents can include TNF receptor superfamily cytokine agonists or cytoline agonist antibodies. Additional compounds that directly or indirectly increase TNF-alpha can be readily identified using routine screening assays for TNF-alpha expression levels or activity. Desirably, an inducer of T-lymphopenia also promotes organ formation, promotes differentiation of donor cells (e.g., blood cells) into a desired cell type, and/or induces damage to host cells of a predetermined cell type to facilitate incorporation of donor cells into the desired organ. In some embodiments, transient T-lymphopenia is induced for a period of time sufficient to destroy at least 10, 20, 30, 40, 50, 60, 80, 90, 95, or 100% of the autoimmune cells in the patient (e.g., B-cells that produce a self-reacting antibody, T-cells that are activated by presented self epitopes, or a subset of antigen presenting cells with defective antigen presentation). In some embodiments, that agent that kills naive T-cells is not BCG or FAS.

Other agents that can be administered to a mammal to induce T-lymphopenia include IL-1, IL-2, IL-4, IL-5, IL-6, IL-7, IL-10, IL-11, IL-12, IL-13, IL-18, INF-alpha, IFN-beta, IFN-gamma, TFG-beta, PDGF, and/or VEGF, as well as a small molecule or antibody agonist of TLR1, TLR2, TLR6, TLR3, TLR4, TLR5, TLR7, and/or TLR9. In addition to BCG, biologics of diverse compositions such as BCG, BLP, fibronectin Domain A, lipoarabinomannan, LPS binding protein, LPS, lipoteichoic acid, macrophage stimulatory lipopeptide 2, mannosylated phosphatidylinositol peptidoglycan, respiratory syncytial virus protein F, and soluble tuberculosis factor may also be administered to induce T-lymphopenia, if desired.

Any of the methods of the present invention can be used to treat a mammal (e.g., a human patient) who has an autoimmune disease, for example, diabetes, immunologically-mediated glomerulonephritis, chronic hepatitis, primary biliary cirrhosis, or primary sclerosing cholangitis.

In another aspect, the invention features an isolated Hox11-expressing pluripotent cell population, where at least 75% of the population is CD45(−). In one embodiment, as least 90% of the cell population is CD45(−). In another embodiment, the cell population contains one or more cell surface markers selected from the group consisting of: retinoic acid receptor, estrogen receptor, EGF receptor, CD49b, VLA2, CD41, LFA-1, ITB2, CD29, NTC3 receptor, plasminogen receptor, transferrin receptor, TGF receptor, TNF receptor (e.g., TNF receptor 1), PDGF receptor, thyroid growth hormone receptor, and integrin beta 5. The isolation of a cell population enriched in cells that do not express CD45 is performed as previously described herein. Preferably, the pluripotent cell population is purified spleen cell population. The cell population can be further purified according to standard affinity techniques known in the art using one or more of the cell surface markers described above.

In another aspect, the invention features an isolated CD45(−) pluripotent cell population, where at least 75% of the population expresses Hox11. In one embodiment, as least 90% of the cell population expresses Hox11. In another embodiment, the cell population contains one or more cell surface markers selected from the group consisting of: retinoic acid receptor, estrogen receptor, EGF receptor, CD49b, VLA2, CD41, LFA-1, ITB2, CD29, NTC3 receptor, plasminogen receptor, transferrin receptor, TNF receptor (e.g., TNF receptor 1), receptor, PDGF receptor, thyroid growth hormone receptor, and integrin beta 5. The isolation of a cell population enriched in cells that do not express CD45 is performed as previously described herein. Preferably, the pluripotent cell population is purified spleen cell population. The cell population can be further purified according to standard affinity techniques known in the art using one or more of the cell surface markers described above.

In yet another aspect, the invention features a pluripotent cell transfected with a Hox11 gene, preferably a human Hox11 gene, where the cell is capable of differentiating into a cell selected from the group consisting of: a pancreatic cell, a spleen cell, a liver cell, a kidney cell, a nerve cell, and a bone cell. In preferred embodiments, the cell is derived from cord blood or the spleen. In a most preferred embodiment, the cell does not express CD45.

### DEFINITIONS

By “autoimmune disease” is meant a disease in which an immune system response is generated against self epitopes. Examples of autoimmune diseases that can be treated using the AGM cells of the invention include alopecia areata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, systemic lupus erythmatosous, ulcerative colitis, psoriatic arthritis, essential mixed cryoglobulinemia, fibromyalgia-fibromyositis, Graves' disease, Guillain-Barré, Hashimoto's thyroiditis, hypothyroidism, idiopathic pulmonary fibrosis, idiopathic thrombocytopenia purpura (ITP), IgA nephropathy, insulin dependent diabetes, juvenile arthritis, lichen planus, lupus, Ménière's disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, Stiff-Man syndrome, Takayasu arteritis, temporal arteritis/giant cell arteritis, ulcerative colitis, uveitis, vasculitis, vitiligo, and Wegener's granulomatosis.

In the present invention, MHC class I and peptide complexes can be used for the restoration of T-cell selection by the elimination of pathogenic (e.g., autoimmune) T cells. As used herein, the term “MHC class I and peptide” includes naturally occurring complexes (i.e., MHC complexes with native antigen-derived peptides) and complexes with peptides that differ from native antigen-derived peptides but which are nonetheless able to form a complex with class I that is effective to maintain functional cells according to the invention. Exemplary peptides that differ from native antigen-derived peptides may contain unnatural amino acids, e.g., D-amino acids, as well as naturally-occurring amino acids. Useful MHC peptide complexes include those that are linked complexes for crosslinking more than one host T cell receptor. Linked complexes may have higher affinity and thus be more effective in the removal of autoreactive T cells. MHC class I and peptide complexes can also include MHC fragments composed of the exterior binding groove but with removed or altered conserved regions. MHC class I and self peptide complexes have been crystallized and the resulting crystalline structure used to form soluble compounds with binding to the T cell receptor that is identical to or approximates that of the native complex. These soluble compounds can also be used in the methods of the invention.

Preferred MHC class I and peptide complexes are those in which a chain of amino acids between 8 and 10 residues in length is correctly complexed with an MHC class I molecule that is either semi-allogeneic, i.e., at least one MHC class I allele is mismatched and at least one MHC class I allele is matched between donor and recipient, or syngeneic, i.e., all MHC class I alleles are matched between donor and recipient, where the MHC class I and peptide complex contributes to the re-education or re-selection of the immune system.

MHC class I and self peptide complexes can be harvested from normal lymphoid cells. Alternatively MHC class I and self peptide complexes can be expressed in E. coli or eukaryotic cells and then refolded with antigenic peptides in vitro prior to administration. In some embodiments, the MHC class I and peptide are present on the surface of cells that are administered to the patient. Other MHC class I and peptide complexes are soluble complexes that are not expressed on the surface of a cell. In particular embodiments, the extracellular region of MHC class I (e.g., a Fab fragment of MHC class I) or soluble, full-length MHC class I is incubated with one or more peptides according to known methods under conditions that allow a peptide to bind the MHC class I fragment, and the resulting MHC class I and peptide complex is administered to the patient. In other embodiments, a mixture of MHC class I and peptide are administered to the patient, and the MHC class I and peptide bind in vivo after administration to the patient or multiple MHC class I and peptide complexes are administered. In some embodiments, the administered MHC class I has 1, 2, 3, or 4 alleles with at least 60, 70, 80, 90, 95, or 100% sequence identity to that MHC class I expressed by the patient. Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Wis. 53705). This software program matches similar sequences by assigning degrees of homology to various substitutions, deletions, and other modifications.

By “CD45(−),” or a “cell that does not express CD45” is meant a cell that has does not express the CD45 receptor by immunofluorescence analysis, or a population of cells in which at least 75% of the cells (such as, for example 80%, 90%, or 95%) do not express the CD45 receptor by immunofluorescence analysis.

By “functional cell,” is meant cells that carry out their normal in vivo activity. In certain desirable embodiments of the invention, the cells are capable of expressing endogenous self-peptide in the context of MHC class I.

By “Hox11-expressing cell” is meant a cell that expresses Hox11 by RNA analysis, or a population of cells in which at least 75% of the cells (such as, for example 80%, 90%, or 95%) expresses Hox11 by RNA analysis.

By “predetermined type,” when used in reference to functional cells, is meant a defined cell type. For example, one skilled in the art may decide to carry out the method of the present invention in order to increase or maintain the number of functional islet cells in the pancreas. In this example, the cells of a predetermined type are islet cells or islet precursor cells.

By “predominantly expresses” is meant a cell population in which at least 75% of the cells (such as, for example 80%, 90%, or 95%) expresses the characteristic (e.g., a cell surface marker) that the phrase refers to.

Standard assays can be used to determine whether administered cells form cells of the predetermined cell type in vivo. For example, cells may be analyzed for expression of particular proteins (e.g., proteins specific for the predetermined cell type) using standard Western or immunofluorescence analysis or for the expression of particular mRNA molecules (e.g., mRNA molecules specific for the predetermined cell type) using a cDNA array (Ausubel et al., Current Protocols in Molecular Biology, John Wiley & Sons, New York, 2000). Examples of other characteristics of the administered cells that may be analyzed to determine whether they have been converted into the desired cell type include the size of the cell, cell morphology, volume of cytoplasm, and cell function (e.g., production of insulin or other hormones).

By “semi-allogeneic,” is meant a match of at least one marker, for example, an MHC class allele, between cells of the same type from different individuals of the same species. Desirably at least two or three MHC class I alleles match between the donor and the host. Standard methods may be used to determine whether an MHC class I allele expressed by a donor cell matches an MHC class I allele expressed by the recipient. For example, antibodies specific for a particular MHC class I allele can be used to determine what alleles are expressed. Alternatively, PCR amplification of nucleic acids encoding MHC class I alleles can be used.

By “syngeneic donor cell” or “isogeneic donor cell,” is meant (i) a donor cell that is genetically identical, or matched at the HLA region (i.e., has at least four, and preferably all 6, of the standard markers in common with), to a cell of the recipient or (ii) a donor cell that is re-administered to the same patient from which it was obtained.

A “TNF-alpha inducing agent,” is desirably a compound that results in the expression of endogenous TNF-alpha, enhances secretion of TNF-alpha, or enhances bioavailability or stability of TNF-alpha. However, TNF-alpha agonists, agents that stimulate TNF-alpha signaling or enhance post-receptor TNF-alpha action, or agents that act on pathways that cause accelerated cell death of autoimmune cells, are also included in this definition. Stimulation of TNF-alpha induction (e.g., by administration of CFA) is desirably carried out prior to, after, or during administration (via implantation or injection) of cells in vivo.

Similarly, other “inducing agents” may cause the expression of a gene product, either through activation of a silent gene in an endogenous cell or by the insertion of an exogenous gene into an endogenous cell (e.g., via a gene therapy approach). Gene therapy approaches are known to those skilled in the art (e.g., see U.S. Pat. No. 6,384,202).

By “selectively killing blood cells” is meant directly or indirectly reducing the number or relative percentage of a subpopulation of blood cells (e.g., autoreactive lymphoid cells such as T- or B cells or the defective antigen presenting cells) such as a subpopulation of unstimulated cells or stimulated cells. In desirable embodiments, the subpopulation is a subset of T-cells, B-cells, or macrophages. Desirably, the killed memory T-cells are autoimmune T-cells, i.e., T-cells that are activated by presented self epitopes. In desirable embodiments, the killed naïve T-cells are cells that would otherwise become autoimmune T-cells. Desirably, the number of autoimmune T-cells or cells that would otherwise become autoimmune T-cells decreases by at least 25, 50, 100, 200, or 500% more the number of healthy non-autoimmune T-cells decreases. In some embodiments, the number of autoimmune T-cells or cells that would otherwise become autoimmune T-cells decreases by at least 25, 50, 75, 80, 90, 95, or 100%, as measured using standard methods. The T-cells can be killed due to any pathway, such as apoptosis, necrosis, and/or activation induced cell death. Apoptosis can be assayed by detecting caspase-dependent cell shrinkage, condensation of nuclei, or intranuclear degradation of DNA. Necrosis can be recognized by caspase-independent cell swelling, cellular degradation, or release of cytoplasmic material. Necrosis results in late mitochondrial damage but not cytochrome C release. In some embodiments, memory T-cell are killed by apoptosis, and naive T-cells are killed by necrosis. For the treatment of lupus, B-cells are desirably killed or, alternatively, they are allowed to developmentally mature.

By “stimulated cell,” “stimulated organ,” or “stimulated tissue” is meant a cell (e.g., a memory T-cell, a B-cell, or a macrophage), organ, or tissue, respectively, that has been exposed to an antigen or a ligand for a cell-surface receptor (such as, for example, a ligand for a receptor that induces apoptosis).

By “unstimulated cell,” “unstimulated organ,” or “unstimulated tissue” is meant a cell (e.g., a naïve T-cell, a B-cell, or a macrophage), organ, or tissue, respectively, that has not been exposed to an antigen or a ligand for a cell-surface receptor.

As used herein, the term “totipotent cell” refers to a cell capable of developing into all lineages of cells. Similarly, the term “totipotent population of cells” refers to a composition of cells capable of developing into all lineages of cells. Also as used herein, the term “pluripotent cell” refers to a cell capable of developing into a variety of lineages (albeit not all lineages). A “pluripotent population of cells” refers to a composition of cells capable of developing into less than all lineages of cells. By definition, a totipotent cell or composition of cells is less developed than a pluripotent cell or compositions of cells.

### BRIEF DESCRIPTION OF THE DRAWINGS

FIG. 1 shows the effects of treatment with live or irradiated splenocytes on the restoration of normoglycemia and pancreatic histology in diabetic NOD mice. (A) Kaplan-Meier curves for normoglycemia. Diabetic NOD females were treated with a single injection of CFA and biweekly injections for 40 days of either live (circles) or irradiated (squares) splenocytes from CByB6F1 males. Syngeneic female islets transplanted subrenally at the onset of treatment were removed after either 40 days (left panel) or 120 days (right panel). Blood glucose concentration was monitored at the indicated times after islet graft removal, and the percentage of animals that remained normoglycemic was plotted. Data are from 9 or 8 (left panel) or from 12 or 13 (right panel) animals that received live or irradiated splenocytes, respectively; P=0.0002 (left panel), P=0.68 (right panel) for comparison between the two treatment groups. (B) Pancreatic histology. NOD mice treated with live (right panels) or irradiated (middle panels) splenocytes and subjected to removal of the islet graft after 40 days as described in (A) were killed 80 days after treatment onset or after the return of hyperglycemia, respectively. Pancreatic sections were subjected to staining with hematoxylin-eosin (top panels) or to immunofluorescence analysis with antibodies to CD45 (bottom panels). The pancreas of an untreated NOD female (25 weeks of age) after the onset of mild hyperglycemia is also shown (left panels). The distinctive histological patterns of invasive insulitis (left panels), peri-insulitis (middle panels), and minimal peri-insulitis (right panels) are apparent. Arrows indicate the outline of an islet. Original magnification, ×200. (C) Pancreatic histology. Three NOD mice successfully treated with either irradiated (top panels) or live (bottom panels) splenocytes were killed ˜9 weeks after removal of the 120-day islet graft. Sections of each pancreas were stained with hematoxylin-eosin. Pronounced peri-insulitis was apparent only in the NOD mice treated with irradiated cells. Original magnification, ×200.

FIG. 2 shows a two-color flow cytometric analysis with antibodies specific for H-2K^{d} or H-2K^{b} on PBLs obtained from female NOD mice successfully treated with CFA and either live splenocytes (upper right panel) or irradiated splenocytes (upper left panel) from CByB6F1 males. Subrenal islet transplants were removed after 120 days and blood was collected 12 and 11 weeks after treatment termination, respectively. PBLs from an untreated NOD mouse at 12 weeks of age (lower left panel) and from a normal CByB6F1 mouse at 12 weeks of age (lower right panel) were similarly analyzed for comparison. The percentages of cells expressing both H-2K^{d} and H-2K^{b} are indicated.

FIG. 3 shows long-term restoration of normoglycemia and the direct contribution of donor splenocytes to islet regeneration in successfully treated NOD female mice. (A) Blood glucose concentrations during the lifetime of two NOD females (#789 and #790 in Table 2) successfully treated with CFA and CByB6F1 male splenocytes as well as with a temporary subrenal transplant of syngeneic islets. (B) Immunofluorescence and fluorescence in situ hybridization (FISH) analyses of serial pancreatic sections from the successfully treated NOD females #789 (left panels) and #790 (right panels). The two top panels show immunofluorescence staining of islets with antibodies to insulin; the subsequent three pairs of images show FISH signals obtained with a Y chromosome-specific probe and nuclear staining with DAPI in sections containing islets (arrows), pancreatic ducts (arrowheads), and exocrine pancreas, respectively. (C) Immunofluorescence and FISH analyses of serial pancreatic sections from a C57BL/6 male (left panels) and C57BL/6 female (right panels). The two top panels show immunofluorescence staining of islets with antibodies to insulin; the subsequent three pairs of images show FISH signals obtained with a Y chromosome-specific probe and nuclear staining with DAPI (blue) in sections of the endocrine and exocrine portions of the pancreas as in (B). (D) Representative confocal micrographs obtained from three focal planes (−3, 0, and +3 μm) of a pancreatic section derived from the successfully treated NOD mouse #789. Images show staining with antibodies to insulin and nuclear staining for a large islet surrounded by exocrine tissue. The lower panels are higher magnification views of the fields shown in the upper panels.

FIG. 4 shows two-color FISH analysis of the sex chromosomes of NOD female mice successfully treated with either live or irradiated male splenocytes. (A) Analysis of islet and pancreatic duct chimerism in NOD females successfully treated with live (top panels) or irradiated (bottom panels) CByB6F1 male splenocytes. Pancreatic sections were subjected to nuclear staining with DAPI (blue) and to FISH analysis with a Cy3-conjugated X chromosome-specific probe (red dots) and an FITC-conjugated Y chromosome-specific probe. Purple represents overlap of Cy3 and DAPI signals. Arrows indicate the outlines of islets (B) Control pancreatic sections from an untreated NOD female (left panel) and an untreated NOD male (right panel) stained as in (A). (C) Sections prepared from the brain, liver, and kidney of a NOD female mouse after long-term disease reversal induced by treatment with CFA and live CByB6F1 male splenocytes were stained as in (A).

FIG. 5 shows the identification of Hox11-expressing pluripotent cells in the spleen of adult mice and the effect of treatment with separated CD45⁻ or CD45(+) compared to whole CByB6F1-GFP splenocytes on pancreatic histology in prediabetic NOD mice. (A) Polyadenylated RNA isolated from the pancreas of a C57BL/6 mouse embryo at E15 or from the spleen of adult C57BL/6, CByB6F1, NOD SCID, or NOD mice was subjected to RT-PCR analysis with primers specific for Hox11, Pdx1, or the β-actin gene. The amounts of PCR products derived from Hox11 and Pdx1 mRNAs were determined by densitometry and normalized by the corresponding amount of that derived from β-actin mRNA; the normalized values are shown. (B) Western blot. Pancreatic extracts (2 μg or 5 μg) from control and treated NOD mice probed with anti-GFP antibodies. (C) Prediabetic NOD females (12 weeks old) were treated with CFA and CD45⁻ (left upper panels) CD45(+) (right upper panels) or whole (left lower panels) CByB6F1-GFP splenocytes and were monitored for >120 days. Serial pancreatic sections containing islets identified with insulin and CD45 co-staining were then subjected to immunohistochemical analysis with an anti-GFP antibody. Right panels: Rhodamine filter. (D) Serial pancreatic sections from a diabetic NOD, prediabetic NOD (12 weeks old), C57BL/6 control, prediabetic NOD female treated with CFA and whole CByB6F1 splencoytes, prediabetic NOD female treated with CFA and CD45(+) CByB6F1 splencoytes, and prediabetic NOD female treated with CFA and CD45(−) CByB6F1 splenocytes were subjected to immunofluorescence analysis with antibodies to insulin or to CD45, as indicated; merged images are shown in the bottom row.

FIG. 6 shows the expression of Hox11 in the spleen of adult mice. (A) Polyadenylated RNA isolated from the pancreas of a C57BL/6 mouse embryo at E15 or from the spleen of 12-week-old C57BL/6, CByB6F1, NOD SCID, or NOD mice was subjected to RT-PCR analysis with primers specific for Hox11, Pdx1, or the β-actin gene. The amounts of PCR products derived from Hox11 and Pdx1 mRNAs were determined by densitometry and normalized by the corresponding amount of that derived from β-actin mRNA; the normalized values are shown below each lane. (B) Expression of Hox11 in CD45− splenocytes. Polyadenylated RNA isolated from CD45− or CD45⁺ splenocytes of 12-week-old C57BL/6 mice was analyzed by RT-PCR with primers specific for Hox11 or the β-actin gene. (C) Immunofluorescence analysis of the spleen from a 12-week-old C57BL/6 mouse stained with antibodies to Hox11 (red) and with 4′,6-diamidino-2-phenylindole (blue nuclei), (D) Polyadenylated RNA isolated from PBLs, bone marrow cells (BM), and the spleen of 12-week-old C57BL/6 mice and from the spleen of a C57BL/6 embryo at E15 was subjected to RT-PCR analysis with primers specific for Hox11, or the β-actin gene.

### DETAILED DESCRIPTION

Based on the findings reported herein, the invention features a therapeutic composition containing mammalian, preferably human, post-fetal, AGM cells in a pharmaceutically acceptable carrier, e.g., sterile saline, or a solid or semi-solid (e.g., gel) matrix. The AGM cells of the invention can be administered to a human patient to treat any medical condition that can be improved by the administration of stem cells. AGM cells are so called because they are proposed to be a remnant of an embryonic stem cell region called the aorta gonad mesoderm (AGM), which express developmental proteins indicative of the persistence of a fetal stem cell in an adult animal. The AGM cells are characterized by their expression of Hox11 (i.e., Hox11 is naturally expressed by AGM cells), and by their lack of expression of CD45.

The invention also features a population of cells enriched in mammalian, preferably human, AGM cells; by “enriched” is meant that the population of cells is made up of at least 1%, preferably 2%, 5% or 10%, more preferably 15%, 20%, 25%, or 30%, and most preferably 35%, 40%, 45%, or 50% or more post-fetal AGM cells.

The invention also features methods for treating the medical conditions recited herein by administering to the patient to be treated an agent or combination of agents that mobilizes the AGM cells residing in the patient's body (e.g., the spleen, tonsils, adenoids, and thymus).

Patients who potentially can benefit from the administration of AGM cells fall broadly into three classes:
1) patients suffering from autoimmune diseases,
2) patients in need of organ or cell regeneration for reasons other than treatment of autoimmune disease, and
3) patients in need of immune, especially hematopoietic, reconstitution.

The first category of patients, those suffering from autoimmune diseases, such as, e.g., Type I diabetes, generally can benefit from the regeneration of cells or organs that have been wholly or partially destroyed by autoimmune attack, e.g., the insulin-producing islet cells of the pancreas which have been destroyed in Type I diabetes patients. Other autoimmune disease in which regeneration can be effected by AGM cells of the invention include, but are not limited to, alopecia areata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, systemic lupus erythmatosous, ulcerative colitis, psoriatic arthritis, essential mixed cryoglobulinemia, fibromyalgia-fibromyositis, Graves' disease, Guillain-Barré, Hashimoto's thyroiditis, hypothyroidism, idiopathic pulmonary fibrosis, idiopathic thrombocytopenia purpura (ITP), IgA nephropathy, insulin dependent diabetes, juvenile arthritis, lichen planus, lupus, Ménière's disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, Stiff-Man syndrome, Takayasu arteritis, temporal arteritis/giant cell arteritis, ulcerative colitis, uveitis, vasculitis, vitiligo, and Wegener's granulomatosis.

The administration of AGM cells can also benefit patients in need of organ or cell regeneration due to a wide range of medical conditions. These patients include those who have suffered medical or accidental trauma, such as patients who have suffered heart attack or stroke, or brain or other organ injuries due to accident-related trauma, and patients suffering from degenerative diseases such as liver, kidney, or heart failure, or neurodegenerative diseases such as Alzheimer's disease, Parkinson's disease, and amytrophic lateral sclerosis.

AGM cells can also be administered to patients in need of hematopoietic reconstitution, e.g., those patients whose bone marrow and other immune system components have been damaged or destroyed by chemotherapy and/or radiation treatment of cancer.

AGM cells of the invention can be prepared for therapeutic administration according to methods analogous to those used to prepare other stem cell types for administration to patient. The AGM cells can be derived from the patient's own body (e.g., the spleen, tonsils, adenoids, and thymus of the patient), or from the body of a recently-deceased donor, e.g., an accident victim (e.g., the spleen, tonsils, adenoids, and thymus of a donor). In either case, the most AGM cell-rich region of the spleen is the capsule, the cells of which can readily be dissected away from the rest of the spleen. AGM cells can also be isolated from the pulp of the spleen, and from the tonsils, adenoids, and thymus.

The AGM cells present in the spleen, tonsils, adenoids, and thymus are mixed with non-AGM cells, such as non-AGM splenocytes. In the therapeutic methods of the invention, it is not necessary to completely purify the AGM cells away from other non-AGM cells to form a homogeneous population of AGM cells. It can be advantageous however, to enrich the cells used in therapy for AGM cells. This can be accomplished by first obtaining cells from the spleen, tonsils, adenoids, or thymus and then enriching the cell sample for non-lymphoid cells, based on cell surface markers such as CD45, which AGM cells lack. Preferably, the AGM cells are obtained from the capsule of the spleen, although AGM cells can also be isolated from the pulp of the spleen, and from the tonsils, adenoids, and thymus. Further enrichment can be achieved based on the presence or absence of other markers expressed on the cell surface of AGM cells (see, e.g., Tables 6 and 9, which list cell surface markers expressed by AGM cells). If desired, prior to therapy, the isolated cells, including AGM cells, can be expanded in number, and the desired cells selected, using known techniques, such as those described in Kraus et al., U.S. Pat. No. 6,338,942, hereby incorporated by reference. In an embodiment, the AGM cells can be expanded in the presence of an agent, e.g., a growth factor or an agonist (e.g., a TNF receptor 1 agonist).

Treatment of a patient with a disease state amenable to treatment with AGM cells can involve, e.g., intravenous infusion of a composition containing AGM cells, e.g., a composition containing between about 1×10⁴ AGM cells to about 1×10¹⁰ AGM cells. In an embodiment, the composition contains at least about 1×10⁷ AGM cells. The cells can be substantially pure AGM cells, but more typically will consist of a heterogenous population of cells composed of at least 1%, preferably 2%, 5% or 10%, more preferably 15%, 20%, 25%, or 30%, and most preferably 35%, 40%, 45%, or 50% or more post-fetal AGM cells mixed with other cell types including non-AGM cells and, optionally, other cell types as well, e.g., bone marrow cells. Preferred compositions of the invention include those having at least about 25%, and more preferably at least about 45% or more, AGM cells. Chemical agents can be administered as well with the cells, e.g., an agent that kills or inactivates pathologic T-cell, e.g., live or killed Bacillus Clamette-Guerin (BCG), or an immune adjuvant such as Complete Freund's Adjuvant (CFA), as is discussed herein.

For the treatment of some medical conditions, rather than intravenous administration, in situ administration is desirable. For example, in treating a neurodegenerative disease, such as Parkinson's Disease, administration of AGM cells directly to the affected region of the brain may be preferred.

The invention also relates to repairing and regenerating damaged tissue in a mammal (e.g., a human patient) by administering a composition that includes the AGM cells. Such damage may result from an existing autoimmune disease, or may be the result of a non-autoimmune insult. I have previously shown that eliminating autoimmune cells and re-educating the immune system are important components of an effective treatment of an autoimmune disease (described in U.S. patent application Ser. Nos. 10/358,664, 09/521,064, 09/768,769, and Ryu et al., Journal of Clinical Investigations, 108: 31-33, 2001, which are hereby incorporated by reference herein).

The invention also includes the use of allogeneic and xenogeneic AGM cells and tissues, in addition to syngeneic and autogeneic cells and tissues. I have previously described a transplantation method to introduce allogeneic and xenogeneic tissues into non-immunosuppressed hosts in which the cells are modified such that the donor antigens are disguised from the host's immune system (U.S. Pat. No. 5,283,058; and Markmann et al., Transplantation 54: 1085-89, 1992, each of which are hereby incorporated by reference). It has also been shown that a brief two-component therapy is able both to reestablish self-tolerance and to eliminate selectively the pathological memory T cells by the induction of apoptosis (see, e.g., Ryu et al., Journal of Clinical Investigations, 108: 31-33, 2001, which is hereby incorporated by reference). Simultaneous treatment with TNF-α (or an inducer of endogenous TNF-α production) and with AGM cells of the invention that are partially or fully matched with regard to MHC class I antigens (to reselect pathogenic naïve T cells) thus results in amelioration of the disease condition (e.g., permanent reversal of established diabetes). In the case of Type I diabetes, for example, this method results in the reappearance of insulin-secreting islets in the pancreas of treated subjects that are able to control blood glucose concentration in a manner indistinguishable from that apparent in normal subjects. This methodology can be applied to the treatment of other disease conditions, such as the ones disclosed herein.

### Use of AGM Cells to Treat Type I Diabetes

Compositions of the invention containing AGM cells can be used to treat Type I diabetes in a subject, e.g., a human, in need thereof. The efficacy of using AGM cells to treat Type I diabetes has been successfully demonstrated in mouse model of the disease that correlates well with treatment of the disease in humans. The data support the use of AGM cells, e.g., human AGM cells, in cell therapy of Type I diabetes.

The treatment protocol for the restoration of near-normal pancreatic islet histology and long-term reversal of hyperglycemia in adult diabetic NOD mice (H-2K^{d}D^{b}) has been optimized since its initial description (Ryu et al., Journal of Clinical Investigations, 108: 31-33, 2001) to include both a 40-day regimen of biweekly injections of AGM cells either partially (C57BL/6; H-2K^{b}D^{b}) or fully (CByF1B6F₁/J (CByB6F1); H-2 K^{b}K^{d}D^{b}D^{d}) matched for MHC class I antigens as well as either the repeated administration of TNF-α or a single injection of complete Freund's adjuvant (CFA), the latter of which induces the production of endogenous TNF-α and other cytokines. Preferably, the donor AGM cells are matched for at least one MHC class I molecule, and more preferably with two matching MHC class I alleles. The efficacy of this therapy can also be increased by concomitant euglycemia, which can be achieved by a transplant of syngeneic islets, an intraperitoneal transplant of alginate-encapsulated islets, or by the appropriate administration of insulin.

The reversal of autoimmunity in end-stage diabetic NOD mice was accompanied by the reappearance of functional islets in the pancreas. Female NOD mice treated with live semiallogeneic splenocytes manifested the stable transdifferentiation of male spleen-derived cells into mature islet parenchymal cells. No evidence of engraftment, transdifferentiation, or fusion of male splenocytes in organs including the brain, liver, and kidney was observed, suggesting that, in addition to the low level of hematopoetic chimerism observed, the marked incorporation of donor cells is selective for the diseased pancreas.

Therefore, I have found that, in addition to their contribution to the reversal of autoimmunity, donor AGM cells can also contribute directly to the regeneration of pancreatic islets in a NOD mouse host by manifesting the stable transdifferentiation of spleen-derived AGM cells from an adult into mature islet parenchymal cells, resulting in a treatment protocol for diabetes in a mammal whose successful outcome is solely due to insulin secretion from the host pancreas.

I have also found that AGM cells mediate the education of naïve T cells through the presentation of self antigens and undergo differentiation into, among other cell types, islet cells.

AGM cells can also differentiate into endothelial and endodermal cell types. The pancreas is formed after embryonic day (E) 9.5 in the mouse as a result of the proliferation of endodermal epithelial cells and the invasion by these cells of the surrounding mesenchyme. The adjacent spleen is derived from splanchnic mesoderm, and expression of the Hox11 homeobox gene is obligatory for the differentiation of splenic AGM cells (Roberts et al., Nature 368: 747-9, 1994). Mice deficient in Hox11 lack a spleen; the corresponding stem cells alter their differentiation pathway and contribute instead to pancreatic development (Kanzler et al., Dev. Biol. 234: 231-43, 2001).

Data presented herein show that diabetic NOD mice treated with irradiated AGM cells exhibit long-term restoration of normoglycemia, but with markedly slower kinetics than those apparent in the NOD animals treated with live AGM cells, suggesting that adult diabetic NOD mice contain endogenous precursor cells capable of giving rise to new syngeneic islet structures after the underlying autoimmune disease is eliminated.

Data also presented herein indicate that donor CD45(+) splenocytes, although essential for disease reversal as a result of their contribution of MHC class I and self peptide complexes, do not contain cells able to participate directly in islet generation.

It is therefore proposed that the rapid regeneration of islet cells in diabetic NOD mice treated with live AGM cells, compared with the slower islet regeneration dependent on endogenous cells (apparent in NOD hosts that receive irradiated cells), is due to the mobilization of AGM cells present in the donor spleen cells, and that live donor splenocytes thus not only contributed to reversal of autoimmunity, presumably by mediating the education of naïve T cells through presentation of self antigens, but also provided cells (Hox11⁺CD45(−) AGM cells) that undergo differentiation into islet cells.

The studies with the NOD mouse described in the present examples confirm the efficacy of using AGM cells to treat diabetes and other autoimmune diseases in humans. The ability of an exogenous population of adult spleen cells (i.e., AGM cells) to correct established diabetes permanently, as well as the presence of an endogenous population of NOD mouse stem cells able to give rise to new islets, indicates that therapies to reverse autoimmune diabetes need not incorporate transplantation of exogenous adult islets. In addition to pancreatic islet cells, AGM cells also demonstrate the ability to restore and regenerate a diverse array of other tissues within the body, e.g., pancreatic duct cells, salivary glands, and neuronal cells, as is discussed in the examples below.

### Materials and Methods

Animals, Cells, and Disease Treatment

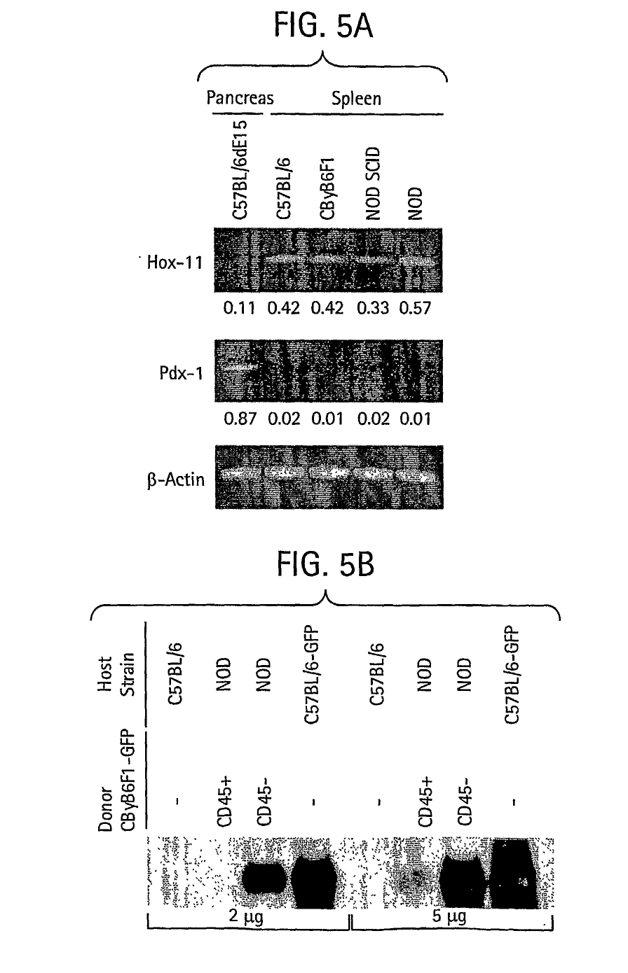

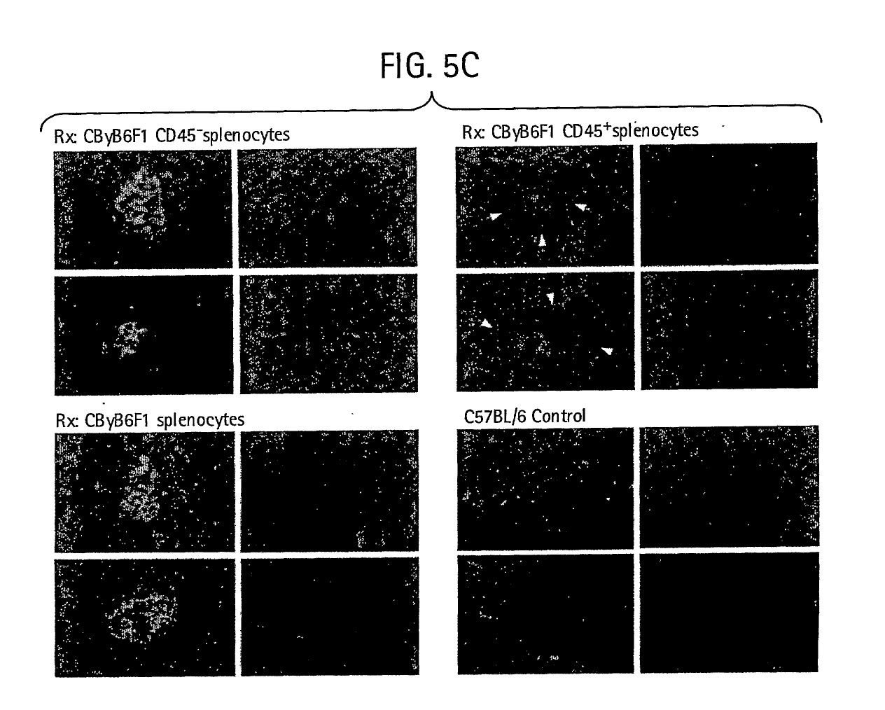

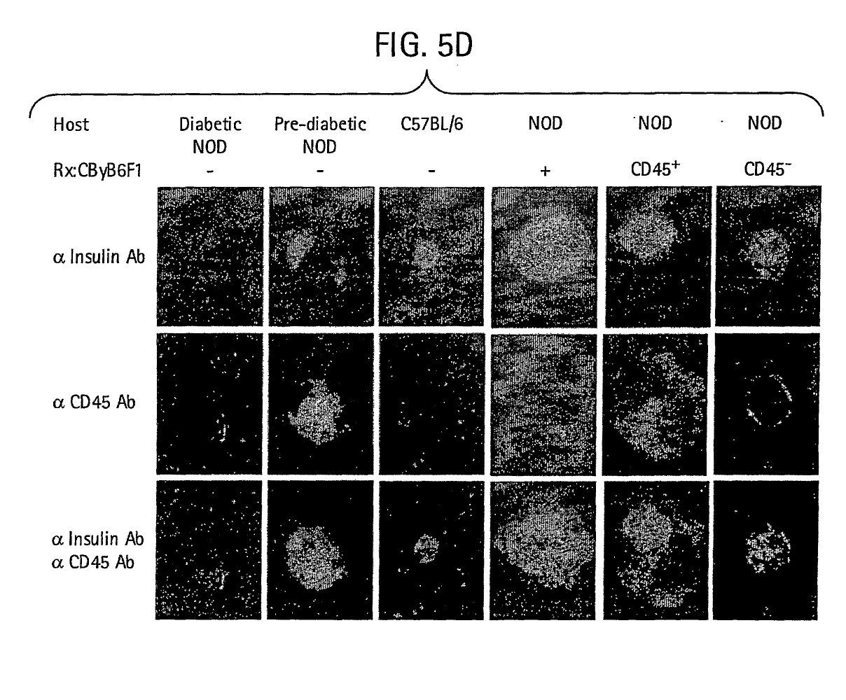

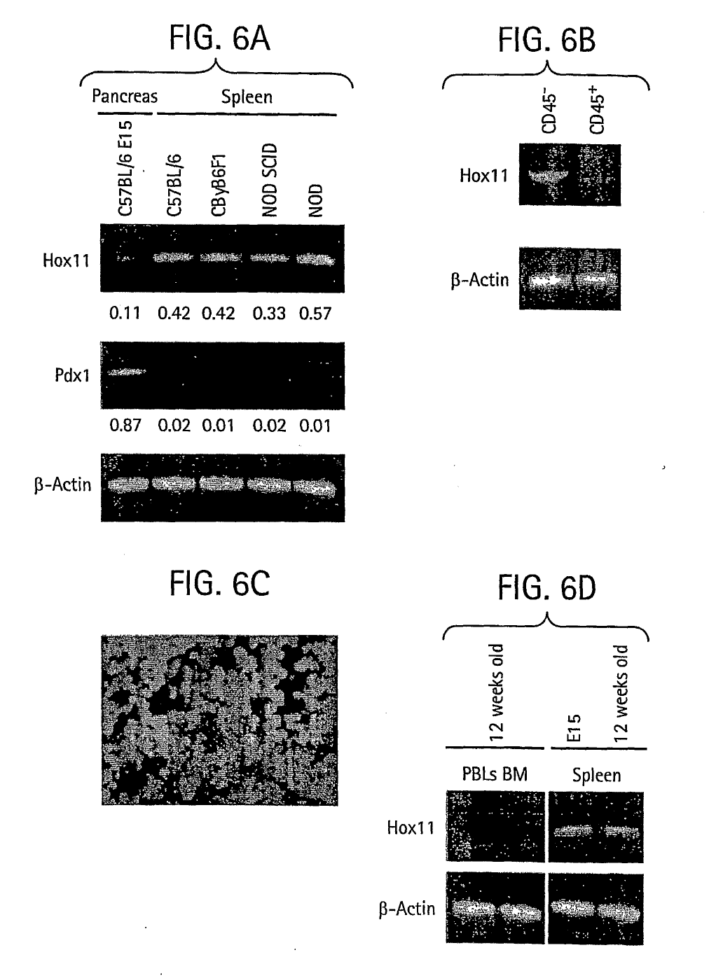

NOD female mice (Taconic Farms, Germantown, N.Y.) as well as male CByF1B6F₁/J (CByB6F1) mice (The Jackson Laboratory, Bar Harbor, Me.) were maintained under pathogen-free conditions. NOD females were screened for the onset of diabetes by the monitoring of body weight and blood glucose, with the diagnosis of diabetes after weight loss accompanied by two consecutive blood glucose concentrations of >400 mg/dL. Diabetes occurred in ˜80% of females by 40 weeks of age in the NOD colony during the present study. At the end stage of diabetes, pancreatic histology revealed the almost complete elimination of identifiable islet structures, as well as elimination of clusters of insulitis that might obscure underlying damaged islets (Table 1, FIG. 5D).

Splenocytes for treatment of NOD females were derived from CByB6F1 (H-2K^{b}K^{d}D^{b}D^{d}) male mice. For irradiation, splenocytes were subjected to 30 Gy of ionizing radiation from a ¹³⁷Cs source. Splenocytes (9×10⁶) were injected into NOD recipients (H-2K^{d}D^{b}) through the tail vein twice a week for 40 days. Complete Freund's Adjuvant (CFA, Difco, Detroit, Mich.) was freshly mixed with an equal volume of physiological saline and then injected (50 μL) into each hind foot pad at the time of islet transplantation or simultaneously with the first splenocyte injection. The induction of endogenous TNF-α by CFA is as effective as is direct TNF-α administration in this model.

The separation of CD45(+) and CD45(−) spleen cells from CByB6F1 donor mice was achieved by isolation of the former cells with the use of mouse-specific CD45 MicroBeads (Miltenyi Biotec, Auburn, Calif.) from total spleen tissue that was mechanically teased apart with forceps. The CD45(+) or CD45(−) splenocytes (4×10⁵ to 5×10⁵) were injected into prediabetic NOD females twice a week for 2 weeks. The recipients also received a single injection of CFA and their blood glucose concentrations were monitored for 120 days or 17 weeks.

Enhanced green fluorescent protein (GFP)-transgenic male (+/−) mice (C57BL/6-TgH(ACTbEGFP)10sb) were purchased (The Jackson Laboratory, Bar Harbor, Me.) and breed to BALB/c female mice to produce some male F1 offspring of the CByB6F1-GFP genotype. GFP transgenic mouse with an “enhanced” GFP cDNA under the control of a chicken beta-actin promoter and cytomegalovirus enhancer make most but not all tissues appear green with only excitation light (JaxMice Data Sheet, Bar Harbor, Me.). Islets of Langerhans for GFP illumination require anti-GFP antibodies for immunohistochemistry and had minimal autofluorescence with excitation.

Islet Transplantation

Islet transplantation for the temporary maintenance of normoglycemia was performed by surgical implantation, beneath the left renal capsule, of 650 syngeneic islets freshly isolated from young (5 to 7 weeks of age) prediabetic NOD females. The exogenous islets were removed by unilateral nephrectomy.

The glucose concentration of orbital blood from non-fasted animals was monitored two to three times a week after transplantation with a Glucometer Elite instrument (Bayer, Mishawaka, Ind.), and transplantation was considered successful if the glucose concentration was reduced to <150 mg/dL within 24 hours after surgery. Body weight was also monitored two to three times a week. Islet grafts were considered to have been rejected if the blood glucose concentration had increased to >250 mg/dL on two consecutive occasions.

Flow Cytometry

Spleens were gently minced on a stainless steel sieve and the resulting spleen cell suspensions as well as heparinized blood collected from the orbital vein were rendered free of red blood cells by a 5-min. exposure to a solution containing 0.83% NH₄Cl. Lymphocytes were then stained with phycoerythrin-conjugated mouse monoclonal antibodies to H-2K^{b} and with FITC-conjugated mouse monoclonal antibodies to H-2K^{d} (Becton-Dickinson, San Diego, Calif.), after which they (>10,000 cells per sample) were subjected to flow cytometry with a FACScan instrument (Becton-Dickinson).

Histology and Immunofluorescence Staining

NOD mice were sacrificed by cervical dislocation and the pancreata were immediately removed and fixed for preparation of paraffin-embedded or cryopreserved sections. Serial sections of from 5 μm to 15 μm were fixed with formalin (10%) for 1 hour for hematoxylin-eosin staining, or with acetone (100%) for 10 min. at 4° C. for immunofluorescence analysis, and were then washed three times with phosphate-buffered saline (PBS). After incubation for 30 minutes with 5% mouse serum in PBS to prevent nonspecific binding of antibodies, the sections were stained for 2 hours with a rat monoclonal antibody to mouse CD45 (1:25 dilution) (NeoMarkers, Fremont, Calif.) or with polyclonal guinea pig antibodies to insulin (1:100) (Linco, St. Charles, Md.) or polyclonal rabbit antibody to anti-GFP (1:50) (Abcan Limited, Cambridge, UK); the antibody to CD45 reacts with all murine isoforms and allelic variants of CD45. The slides were then washed three times for 5 min. with PBS, incubated for 1 hour with FITC- or Texas red-conjugated goat secondary antibodies (Jackson ImmunoResearch Laboratories, West Grove, Pa.), and then washed again three times for 5 min. with PBS. Coverslips were applied with Vectashield mounting medium (Vector, Burlingame, Calif.) and the slides were examined with a fluorescence microscope. All fluorescence was evaluated both with a match and irrelevant filter to the label to prove the specificity of the signal.

### FISH and Confocal Microscopic Analysis

Single- and double-label fluorescence in situ hybridization (FISH) analyses were performed as described (Schwartz et al., J. Clin. Invest. 109: 1291-302; 2002). Whole organs, including the pancreas, brain, liver, and kidney, were immersed in OCT compound and then frozen at −80° C. Serial frozen sections (thickness, 5 μm) were cut, fixed with a mixture of methanol and acetic acid (3:1, vol/vol) for 90 min., dried in air, and dehydrated by exposure to a graded series of ethanol solutions. They were again dried in air, incubated in 70% formamide at 65° C. for 90 s. to 120 s., exposed to ice-cold 70% ethanol, and dehydrated with the graded series of ethanol solutions.

Nucleotide probes were individually denatured by incubation at 65° C. for 10 min. and then at 37° C. for 60 min. to 90 min. One (15 μL) or two (30 μL) probes were added to each slide, which was then covered with a 22 by 32 mm coverslip and sealed with frame fixative (Eppendorf, Westbury, N.Y.). After hybridization overnight at 42° C., the coverslip was removed, and, for detection of the Y chromosome alone, the biotin-conjugated probe (Cambio, Cambridge, UK) was visualized with Texas red-conjugated streptavidin. For detection of both Y and X chromosomes within the same nucleus, the corresponding probes were linked to FITC and Cy3, respectively. Nuclei were also stained with DAPI.

FISH analysis yields an undercount of Y chromosome-positive nuclei as a result of partial nuclear sampling in tissue sections. Although the thin tissue sections used for this analysis prevent false positives due to overlapping nuclear signals, they result in some nuclei (20%) in control male tissue sections being devoid of a detectable Y chromosome. The data shown in Table 2 for the percentages of Y chromosome-positive cells have thus been normalized by a correction factor of 0.8.

Confocal microscopy was performed with a Radiance 2100 instrument equipped with a Multi-Photon system (Bio-Rad, Hercules, Calif.). Fluorescence was excited at 488 nm and emission was monitored at >515 nm. Nuclear size was assessed by NIH image software (version 1.62).

### RT-PCR and Western Analysis

Polyadenylated RNA was isolated from the pancreas or spleen (including the capsule and trabeculae) of C57BL/6, CByB6F1, NOD, or NOD/LtSz-Prkdc^{scid} (NOD SCID) mice. The latter animals are deficient in B and T cells and exhibit severe combined immune deficiency, with their pancreata thus devoid of insulitis and their spleens lacking most lymphoid cell populations. Complementary DNA synthesized from the isolated RNA by RT was subjected to PCR with primers specific for Pdx1 (CACAAGCTTGCGGCCACA-CAGCTCTAC;GAGGGATCCACACTCTGGGTCCCAGAC), Hox11 (AAG-AAGAAGCCGCGCACATC; GGAGTCGTCAGACCACGGCT) and beta-actin-1 (TAAAACGCAGCTCAGTAACAGTCGG; TGCAATCCTGTGGCATCCA-TGAAAC). One step RT-PCR was performed on spleens and pancreata that were removed and soaked in RNA stabilization reagent (Qiagen Inc., Valencia, Calif.) overnight prior to total RNA extraction using an RNA isolation column (Qiagen Inc., Valencia, Calif.). The template of RNA was fixed at 2 μg for each sample and the reaction mixture was 12.5 mM MgCl₂, 10 mM of each deoxynucleoside triphosphate, 20 mM Tris-Cl (pH 8.7), 7.5 mM (NH₄)₂SO₄, 0.6 μg of each primer, 0.4 μL of RNase inhibitor (Invitrogen, Carlsbad, Calif.) and 2 μL of enzyme mix including reverse transcriptase and DNA polymerase. The amplification protocol comprised initial incubations of 50° C. for 30 min. and then 95° C. for 15 min.; 3 cycles of 94° C. for 1 min., 60° C. for 1 min. (Pdx-1) or 63.9° C. for 1 min. (Hox11) or 66.5° C. for 1 min. (beta-Actin) and 72° C. for 10 min. PCR products were separated by electrophoresis on a 1% Tri/Boric Acid/EDTA (TBE) agarose gel and stained with ethidium bromide.

For detection of GFP proteins in cytoplasmic pancreatic extracts, whole pancreases were placed in liquid nitrogen and then dissolved in 700 μL of phosphate buffered saline (pH 7.4) containing 100 μL of protease inhibitor cocktail (protease inhibitor cocktail III; Calbiochem, San Diego, Calif.), 100 μL of phosphatase inhibitor cocktail 1 and 100 μL of phosphatase inhibitor cocktail 2 (Sigma, St. Louis, Mo.). Lysates were centrifuged for 3 min. at 500×g, and resulting supernatants were used. A total of 2 μg or 5 μg of protein per lane were separated by SDS-PAGE, and transferred onto nitrocellulose membranes. Positive GFP bands detected with anti-GFP antibodies were identified using ECL reagents (Amersham Bioscience, Piscataway, N.J.).

### Administration of AGM Cells

Various delivery systems are known and can be used to administer compositions containing the AGM cells of the invention. Methods of introduction include but are not limited to intradermal, intramuscular, intraperitoneal, intravenous, subcutaneous, intranasal, epidural, and oral routes. The cells may be administered by any convenient route, for example by infusion or bolus injection, and may be administered together with other biologically active agents. Administration can be systemic or local. In addition, it may be desirable to introduce the cells into the central nervous system by any suitable route, including intraventricular and intrathecal injection; intraventricular injection may be facilitated by an intraventricular catheter.

In a specific embodiment, it may be desirable to administer the AGM cells of the invention locally to the area in need of treatment; this may be achieved by, for example, and not by way of limitation, local infusion during surgery, by injection, by means of a catheter, or by means of an implant, said implant being of a porous, non-porous, or gelatinous material, including membranes, such as sialastic membranes, or fibers. In another embodiment, the AGM cells or a cell preparation can be delivered in a vesicle, in particular a liposome (e.g., an encapsulated liposome).

Systemic Infusions

The AGM cells prepared according to the methods of the present invention can be administered by infusion into the patient by, e.g., intracoronary infusion, retrograde venous infusion (see, e.g., Perin and Silva, Curr. Opin. Hematol. 11:399-403, 2004), intraventricular infusion, intracerebroventricular infusion, cerebrospinal infusion, and intracranial infusion.

It is anticipated that human therapy is likely to require multiple infusions of any AGM cell composition prepared according to the methods of the present invention, although one infusion may be sufficient. Several infusions of AGM cells can be administered over time, e.g., one on day one, a second on day five, and a third on day ten. After the initial ten day period, there can be a period of time, e.g., two weeks to 6 months without cell administration, after which time the ten-day administration protocol can be repeated.

Whether administered as a single infusion therapy or multiple infusion therapies, it is likely that the recipient will require immunosuppression. The protocols followed for this will follow the precedents now used in human transplantation for bone marrow replacement (i.e., cell transplantation), with such agents as cyclosporin A and FK506.

Direct Injection

Another possible administration route for the AGM cells prepared according to the methods of the present invention is via direct surgical injection (e.g., intramyocardial or transendocardial injection, intracranial, intracerebral, or intracisternal injection, intramuscular injection, intrahepatic injection, intrasplenic injection, and intrapancreatic injection) into the tissue or region of the body to be treated (e.g., the brain, muscle, heart, liver, spleen, pancreas, and vasculature). This method of administration may also require multiple injections with treatment interruption intervals lasting from 2 weeks to 6 months, or as otherwise determined by the attending physician.

Implantation

Compositions containing the AGM cells of the invention can also be administered by implantation into a patient at the site of disease or injury or at a site that will facilitate treatment of the disease or injury.

### Treatment of Disease

Compositions containing AGM cells of the invention, once prepared according to the methods described herein, can be administered to a patient, e.g. a human patient, whether child or adult, for the treatment of a disease or disorder, e.g., an autoimmune disease or disorder, to promote organ or cell regeneration for reasons other than treatment of autoimmune disease or disorder, and to provide immune, especially hematopoietic, reconstitution. Preferably, the AGM cells are typed for the patient using the standard six transplantation markers. While it is preferable that the cells exhibit a 6/6 match, the use of AGM cells with less than a 6/6 match, e.g., a 5/6 or a 4/6 match, is also envisioned. Any rejection that does occur can be offset by the use of standard immunosuppression therapy, as is described herein, e.g., the administration of cyclosporin A or FK506.

In addition, the administered AGM cells may be natural cells or the AGM cells may be engineered to express various genes that do not negatively alter the effectiveness of the cells in engrafting.

### Gene Therapy

Gene therapy can be used, if desired, to modify the AGM cells prepared according to the methods of the present invention. It is envisioned that the AGM cells can be modified, e.g., with a corrected gene product, and administered to the patient using one or more of the methods described herein to treat or prevent a disease or disorder, e.g., an autoimmune disease or disorder, to promote organ or cell regeneration for reasons other than treatment of autoimmune disease or disorder, and to provide immune, especially hematopoietic, reconstitution.

Several methods are known in the art for altering cells for use in gene therapy. These methods include cell transduction (see, e.g., Beutler, Biol. Blood Marrow Transplant 5:273-276, 1999; Dao, Leukemia 13:1473-1480, 1999; and see generally Morgan et al., Ann. Rev. Biochem. 62:191-217, 1993; Culver et al., Trends Genet. 10: 174-178, 1994; and U.S. Pat. No. 5,399,346 (French et al.)); the use of viral vectors; and the use of non-viral vectors, for example, naked DNA delivered via liposomes, receptor-mediated delivery, calcium phosphate transfection, lipofection, electroporation, particle bombardment (gene gun), microinjection, cell fusion, chromosome-mediated gene transfer, microcell-mediated gene transfer, spheroplast fusion, and pressure-mediated gene delivery.

The technique used should provide for the stable transfer of the gene to the cell, so that the gene is expressible by the cell and preferably heritable and expressible by its cell progeny. For general reviews of the methods of gene therapy, see Goldspiel et al., Clinical Pharmacy 12:488-505, 1993; Wu and Wu, Biotherapy 3:87-95, 1991; Tolstoshev, Ann. Rev. Pharmacol. Toxicol. 32:573-596, 1993; Mulligan, Science 260:926-932, 1993; and Morgan and Anderson, Ann. Rev. Biochem. 62:191-217, 1993. Methods commonly known in the art of recombinant DNA technology that can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

### Use of AGM Cells to Regenerate Tissues and Organs

The AGM cells of the invention or their progeny, can be used in a variety of applications. These include, but are not limited to, transplantation or implantation of the cells either in unattached form or as attached, for example, to a three-dimensional framework, as described herein. Typically, 10² to 10⁹ cells are transplanted in a single procedure, with additional transplants performed as required. The tissue produced according to the methods of the invention can be used to repair or replace damaged or destroyed tissue, to augment existing tissue, to introduce new or altered tissue, to modify artificial prostheses, or to join biological tissues or structures.

If the AGM cells are derived from a heterologous source relative to the recipient subject, concomitant immunosuppression therapy can be administered, e.g., administration of the immunosuppressive agent cyclosporine or FK506. If the AGM cells are derived from an autologous source, administration of the immunosuppressive agent may be unnecessary.

In addition, injection of extracellular matrix prepared from new tissue produced using AGM cells, or their progeny, can be administered to a subject or may be used to further culture cells. Such cells, tissues, and extracellular matrix may serve to repair, replace or augment tissue that has been damaged due to disease or trauma, or that failed to develop normally, or for cosmetic purposes.

A preparation of AGM cells or their progeny can be injected or administered directly to the site where the production of new tissue is desired. For example, and not by way of limitation, the AGM cells may be suspended in a hydrogel solution for injection. Alternatively, the hydrogel solution containing the AGM cells may be allowed to harden, for instance in a mold (e.g., a vascular or tubular tissue construct or in the shape of a desired organ), to form a matrix having AGM cells dispersed therein prior to implantation. Once the matrix has hardened, the cell preparation may be cultured so that the cells are mitotically expanded prior to implantation. A hydrogel is an organic polymer (natural or synthetic) which is cross-linked via covalent, ionic, or hydrogen bonds to create a three-dimensional open-lattice structure, which entraps water molecules to form a gel. Examples of materials which can be used to form a hydrogel include polysaccharides such as alginate and salts thereof, polyphosphazines, and polyacrylates, which are cross-linked ionically, or block polymers such as PLURONICS™ or TETRONICS™ (BASF Corp., Mount Olive, N.Y.), polyethylene oxide-polypropylene glycol block copolymers which are cross-linked by temperature or pH. Methods of synthesis of the hydrogel materials, as well as methods for preparing such hydrogels, are known in the art (see, e.g., U.S. Pat. No. 6,960,617).

Such cell preparations may further comprise one or more other components, including selected extracellular matrix components, such as one or more types of collagen known in the art, and/or growth factors and drugs. Growth factors which may be usefully incorporated into the cell formulation include one or more tissue growth factors known in the art or to be identified in the future, such as but not limited to any member of the TGF-β family, IGF-I and -II, growth hormone, BMPs such as BMP-13, and the like. Alternatively, AGM cells may be genetically engineered to express and produce growth factors. Details on genetic engineering of the cells of the invention are provided herein. Drugs that may be usefully incorporated into the cell preparation include, for example, anti-inflammatory compounds, as well as local anesthetics. Other components that may also be included in the preparation include, for example, buffers to provide appropriate pH and isotonicity, lubricants, viscous materials to retain the cells at or near the site of administration, (e.g., alginates, agars, and plant gums) and other cell types that may produce a desired effect at the site of administration (e.g., enhancement or modification of the formation of tissue or its physicochemical characteristics, support for the viability of the cells, or inhibition of inflammation or rejection).

The AGM cells can be administered directly, and those cells that are capable of differentiating can be induced to differentiate by contact with tissue in vivo or induced to differentiate into a desired or predetermined cell type, e.g., pancreatic tissue (e.g., islet cells), salivary gland, neural tissue, spleen, etc.), using in vitro or ex vivo methods before their administration. Techniques for the differentiation of progenitor cells, such as AGM cells, into cells of a particular phenotype are known in the art, such as those described in U.S. Pat. Nos. 5,486,359; 5,591,625; 5,736,396; 5,811,094; 5,827,740; 5,837,539; 5,908,782; 5,908,784; 5,942,225; 5,965,436; 6,010,696; 6,022,540; 6,087,113; 5,858,390; 5,804,446; 5,846,796; 5,654,186; 6,054,121; 5,827,735; and 5,906,934, which describe the transformation of pluripotent cells. For example, Rodgers et al. (U.S. Pat. No. 6,335,195), describes methods for the ex vivo culturing of hematopoietic and mesenchymal pluripotent cells and the induction of lineage-specific cell proliferation and differentiation by growth in the presence of angiotensinogen, angiotensin I (AI), AI analogues, AI fragments and analogues thereof, angiotensin II (AII), AII analogues, AII fragments or analogues thereof, or AII AT₂-type 2 receptor agonists, either alone or in combination with other growth factors and cytokines. In an embodiment, the cell preparation can be administered to produce pancreatic cells, and in particular pancreatic islet cells, by using, e.g., techniques known in the art (see, e.g., Yang et al., Proc. Nat. Acad. Sci. USA 99: 8078-83, 2002; Zulewski et al., Diabetes 50: 521-33, 2001; and Bonner-Weir et al., Proc. Nat. Acad. Sci. USA 97: 7999-8004, 2001). Art-known techniques can also be used to promote the production of various other cell types upon administration of the co-cultured or co-transplanted cells of the invention, e.g., hepatic cells (see, e.g., Lee et al., Hepatology 40: 1275-1284, 2004), neuronal cells (see, e.g., Thondreau et al., Differentiation 319-322-326, 2004), or endothelial cells (see, e.g., Kassem et al., Basic Clin. Pharmacol. & Toxicol. 95:209-214, 2004; and Pittenger and Martin, Circ. Res. 95:9-20, 2004). Optionally, a differentiating agent may be co-administered or subsequently administered to the subject to promote AGM cell differentiation in vivo.

The AGM cells of the invention, or their progeny, can be used to produce new tissue in vitro, which can then be implanted, transplanted, or otherwise inserted into a site requiring tissue repair, replacement, or augmentation in a subject. The AGM cells, or their progeny, may be inoculated or “seeded” onto a three-dimensional framework or scaffold, and proliferated or grown in vitro to form a living tissue which can be implanted in vivo. The three-dimensional framework may be of any material and/or shape that allows cells to attach to it (or can be modified to allow cells to attach to it) and allows cells to grow in more than one layer. A number of different materials may be used to form the matrix, including but not limited to: nylon (polyamides), dacron (polyesters), polystyrene, polypropylene, polyacrylates, polyvinyl compounds (e.g., polyvinylchloride), polycarbonate (PVC), polytetrafluorethylene (PTFE, teflon), thermanox (TPX), nitrocellulose, cotton, polyglycolic acid (PGA), collagen (in the form of sponges, braids, or woven threads, and the like), cat gut sutures, cellulose, gelatin, or other naturally occurring biodegradable materials or synthetic materials, including, for example, a variety of polyhydroxyalkanoates. Any of these materials may be woven into a mesh, for example, to form the three-dimensional framework or scaffold. The pores or spaces in the matrix can be adjusted by one of skill in the art to allow or prevent migration of cells into or through the matrix material. In one example, Naughton et al. (U.S. Pat. No. 6,022,743), describe a tissue culture system in which stem cells or progenitor cells (e.g., cells similar to the AGM cells of the invention) are propagated on three-dimensional supports.

The three-dimensional framework, matrix, hydrogel, and the like, can be molded into a form suitable for the tissue to be replaced or repaired. For example, where a vascular graft is desired, the three-dimensional framework can be molded in the shape of a tubular structure and seeded with the AGM cells, or their progeny. Other cells may also be added to the three-dimensional framework so as to improve the growth of, or alter, one or more characteristics of the new tissue formed thereon. Such cells may include, but are not limited to, fibroblasts, pericytes, macrophages, monocytes, plasma cells, mast cells, and adipocytes, among others.

Alternatively, the AGM cells can be encapsulated in a device or microcapsule, which permits exchange of fluids but prevents cell/cell contact. Transplantation of microencapsulated cells is known in the art, e.g., Balladur et al., Surgery 117: 189-94, 1995; and Dixit et al., Cell Transplantation 1: 275-79, 1992. In one example, the AGM cells may be contained in a device which is viably maintained outside the body as an extracorporeal device. Preferably, the device is connected to the blood circulation system such that the AGM cells can be functionally maintained outside of the body and serve to assist organ failure conditions. In another example, the encapsulated AGM cells may be placed within a specific body compartment such that they remain functional for extended periods of time in the absence or presence of immunosuppressive or immuno-modulatory drugs.

In yet another example, the AGM cells, or their progeny, can be used in conjunction with a three-dimensional culture system in a “bioreactor” to produce tissue constructs which possess critical biochemical, physical and structural properties of native human tissue by culturing the cells and resulting tissue under environmental conditions which are typically experienced by the native tissue. Thus, the three-dimensional culture system may be maintained under intermittent and periodic pressurization and the cells of the invention provided with an adequate supply of nutrients by convection. Maintaining an adequate supply of nutrients to the AGM cells of the invention throughout the generation of a tissue construct having, e.g., approximately 2-5 mm thickness, is important as the apparent density of the construct increases. Pressure facilitates flow of fluid through the three-dimensional construct, thereby improving the supply of nutrients and removal of waste from AGM cells embedded in the construct. The bioreactor may include a number of designs. Typically the culture conditions will include placing a physiological stress on the construct containing AGM cells similar to what will be encountered in vivo. For example, a construct may be cultured under conditions that simulate the pressures and shear forces of blood vessels (see, for example, U.S. Pat. No. 6,121,042, which is hereby incorporated by reference herein).

The invention is further described in the following non-limiting examples.

### EXAMPLES

### Example 1

### The Ability of Live Versus Irradiated Donor Splenocytes to Modulate Autoimmunity

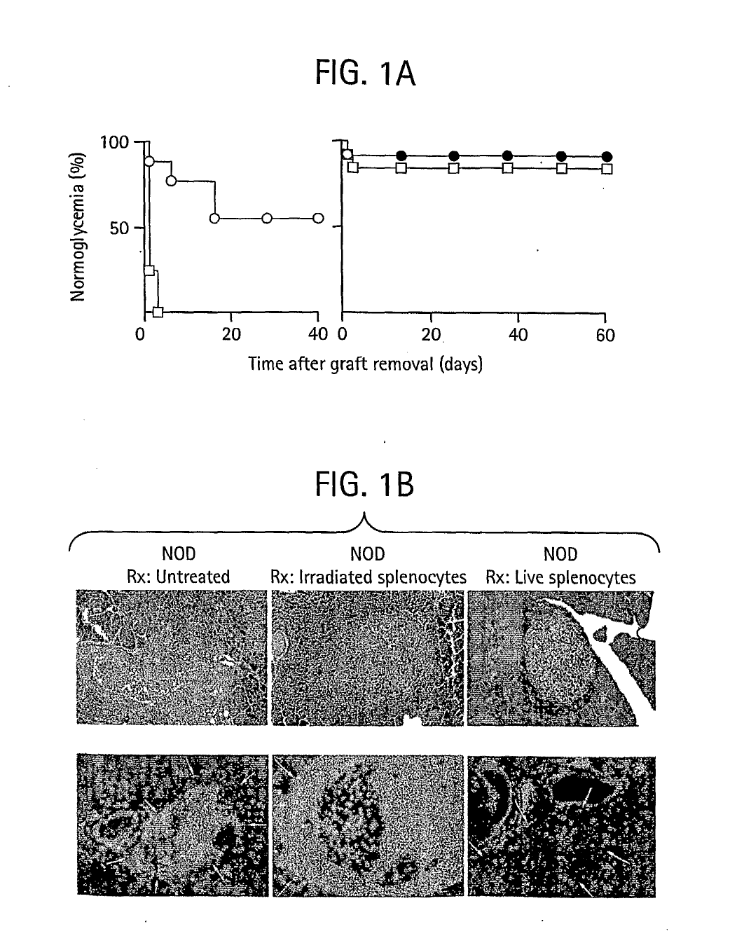

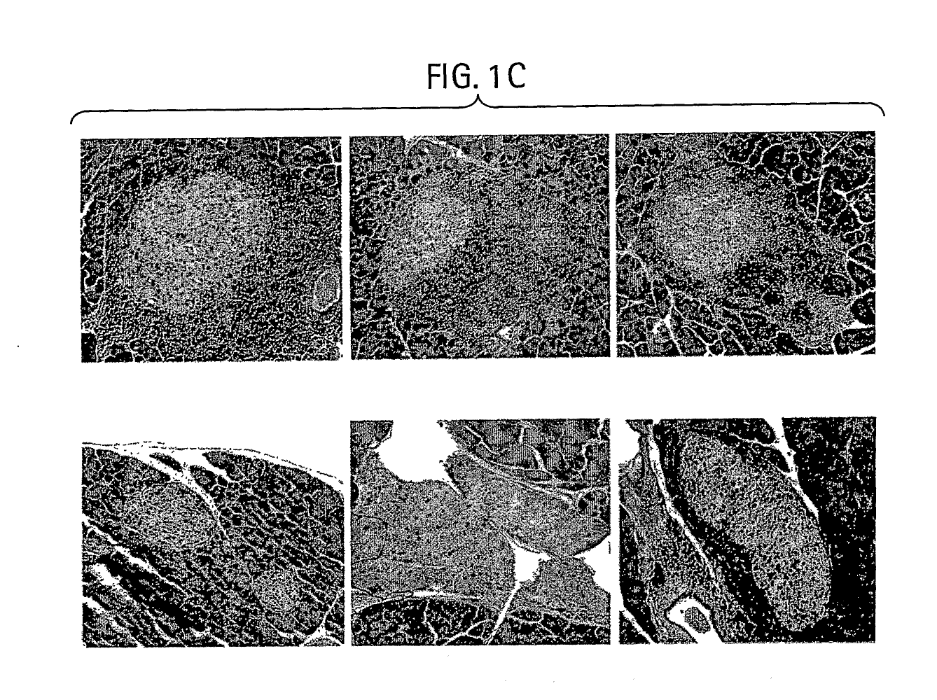

The ability of live versus irradiated donor splenocytes to modulate autoimmunity through selection of naïve T cells was examined as follows. Seventeen severely diabetic NOD females were randomized into two treatment groups that received CFA and either live or irradiated male CByB6F1 mouse splenocytes. This end-stage diabetic state was chosen to ensure both the lack of visible islets, either granulated or nongranulated, and the near-complete elimination of any remaining insulitis that might obscure dead or dying islets, as the pancreata of NOD mice with diabetes of recent onset may still contain scattered islets as well as remaining regions of insulitis (Table 1). Control of blood glucose concentration was achieved with a temporary implant of syngeneic islets under the capsule of one kidney. After the 40-day treatment regimen, the islet transplants were removed by unilateral nephrectomy and blood glucose concentrations were monitored to assess the recovery of endogenous pancreatic islets. Six of the nine NOD mice that received live splenocytes remained normoglycemic (FIG. 1A). In contrast, none of the eight NOD mice that received irradiated splenocytes remained normoglycemia and rapidly developed severe hyperglycemia after removal of the islet implants.

The ability of live or irradiated splenocytes to eliminate invasive insulitis, a sign of active autoimmunity, was assessed by examining the pancreatic histology of the treated animals. The pancreata of NOD mice with restored normoglycemia due to the injection of live splenocytes were removed ˜80 days after the onset of therapy (˜40 days after removal of the islet graft); that of NOD mice treated with irradiated splenocytes were removed after the return of hyperglycemia (˜40 to 45 days after therapy initiation). Consistent with previous observations (Ryu, et al., J. Clin. Invest. 108: 63; 2001), the pancreata of normoglycemic animals treated with live splenocytes contained abundant islets almost uniformly devoid of invasive insulitis (FIG. 1B), with occasional islets associated with small, circumferentially distributed, regions of insulitis (peri-insulitis), a pattern associated with disease non-progression. The pancreata of NOD mice that received irradiated splenocytes also contained islets largely devoid of invasive insulitis, although the number of islets was smaller than that apparent in the animals treated with live splenocytes (FIG. 1B); these islets typically exhibited marked peri-insulitis, as confirmed by immunofluorescence staining with antibodies to CD45 (FIG. 1B), which selectively recognizes all lymphoid (not parenchymal) cells. These histological findings contrast with those observed in a 25-week-old prediabetic NOD mouse that had exhibited an increase (within 2 days) in blood sugar level to 250 mg/dL and whose pancreas exhibited an overall decrease in islet abundance, as well as invasive and peri-insulitis, that obliterated existing islet structure (FIG. 1B). Thus, when examined 40 to 45 days after the initiation of treatment, both live and irradiated splenocytes appeared able to reverse invasive insulitis, with live cells more effective in restoring normoglycemia, as well as inducing the reappearance of abundant pancreatic islets.

TABLE 1
Pancreatic islet histology of untreated NOD female mice.

| | | Islets | | |
|---|---|---|---|---|
| | Blood glucose | | Percentage | No. of insulitis |
| Age (weeks) | (mg/dL) | No. | granulated | clusters* |
| 8 | 115 | 75 | 100 | 154 |
| 12 | 122 | 62 | 100 | 143 |
| 18 | 255 (1x)** | 9 | 15 | 54 |
| 25 | >400 (>2x)* | 1 | 0 | 3 |

*The number of islets counted are overestimates of the actual value because large islets that span adjacent sections are counted more than once. Insulitis clusters were defined either as islets with invasive insulitis or insulitis clusters without visible islet tissue.
**(1X) means this NOD mouse had one blood sugar at 255 at the time of sacrifice: (>2X) means this NOD mouse had two blood sugars greater than 400 mg/dL.

### Example 2

### Kinetics of Pancreatic Islet Recovery

The kinetics of pancreatic islet recovery in additional groups of diabetic NOD mice was examined as follows. Twenty-five new and severely diabetic NOD females were randomized to treatment groups receiving CFA and either live or irradiated male splenocytes and temporary syngeneic islet transplants were maintained for 120 days before nephrectomy to allow a longer period for islet regeneration in situ. Eleven (92%) of the 12 NOD mice that received live splenocytes remained normoglycemic for greater than 26 weeks after disease onset or beyond 52 weeks of age. Moreover, 11 (85%) of the 13 NOD mice that received irradiated splenocytes also remained normoglycemic for greater than 27 weeks after disease onset or beyond 48 weeks of age (FIG. 1A). The longer period of ectopically imposed normoglycemia during treatment greatly increased the frequency of functional islet recovery in both experimental groups, with both live and irradiated splenocytes thus able to contribute to prolonged disease elimination. The average experimental landmark ages are provided in Table 2 for those mice that remained normoglycemic.

TABLE 2
Age at various experimental landmarks of NOD female restored
normoglycemia*

Age (weeks)

| | Live | Irradiated |
|---|---|---|
| | splenocytes | splenocytes |
| | (n = 11) | (n = 11) |
| Diabetes and treatment onset | 26 ± 7 | 22 ± 6 |
| Treatment termination | 31 ± 6 | 28 ± 5 |
| Nephrectomy | 43 ± 3 | 39 ± 2 |
| Analysis of peripheral blood | 43 ± 7 | 39 ± 6 |
| Tissue analysis | 52 ± 7 | 48 ± 6 |

*Data are means ± SE.

The pancreatic histology of mice that had been successfully treated with live or irradiated splenocytes and had experienced persistent normoglycemia for ˜9 weeks after nephrectomy was also examined (Table 3). The pancreata of the NOD mice that received irradiated splenocytes manifested the reappearance of pancreatic islets without invasive insulitis but with pronounced peri-insulitis, as revealed by hematoxylin-eosin staining. In contrast, the pancreata of NOD mice that received live splenocytes exhibited the reappearance of pancreatic islets without invasive insulitis and with minimal or no peri-insulitis (FIG. 1C, Table 3).

TABLE 3
Pancreatic islet histology of successfully treated NOD mice.
Pattern of insulitis (%)

| | Splenocyte | | | | Insulitis |
|---|---|---|---|---|---|
| Animal | treatment | Invasive | Peri | None | magnitude |
| #744 | Live | 2 | 11 | 87 | + |
| #788 | Live | 0 | 28 | 72 | ++ |
| #789 | Live | 0 | 15 | 85 | + |
| #790 | Live | 0 | 22 | 78 | + |
| #838 | Live | 0 | 31 | 69 | + |
| #699 | Irradiated | 43 | 57 | 0 | +++ |
| #703 | Irradiated | 2 | 87 | 11 | +++ |
| #745 | Irradiated | 21 | 58 | 21 | +++ |
| #752 | Irradiated | 44 | 56 | 0 | ++ |
| #754 | Irradiated | 25 | 75 | 1 | +++ |

*Approximately 10 islets were examined for each NOD recipient and the dominant pattern of insulitis was determined for each islet; the predominant extent of insulitis (+, ++, or +++) among the islets of each pancreas is also presented.

### Example 3

### Hematopoetic Chimerism

Lethal preconditioning (such as whole-body irradiation) of a host and introduction of MHC-matched bone marrow cells results in long-term hematopoetic chimerism (Weissman, Science 287: 1442, 2000). To determine whether hematopoetic chimerism also occurred in the non-preconditioned NOD mouse hosts that received live or irradiated splenocytes according to the treatment protocol of Example 4, the peripheral blood lymphocytes (PBLs) of these animals was examined by flow cytometry at mean ages of 43 and 39 weeks, respectively, ˜17 weeks after diabetes onset and >11 weeks after the last injection of donor splenocytes (Table 2). Blood was obtained from the orbital vein, thereby allowing the mice to live after its collection.

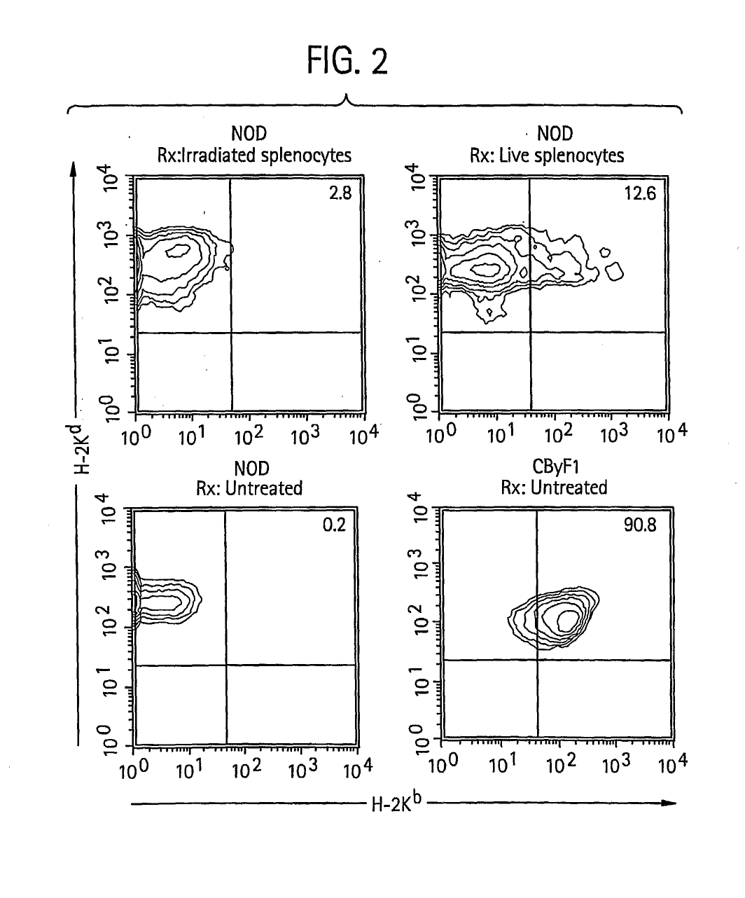

The PBLs from NOD mice (H-2K^{d}) with disease reversal were examined for remaining live CByB6F1 donor cells (H-2K^{b}K^{d}) with allele-specific antibodies to the H-2K^{b} or H-2K^{d} MHC class I proteins. The results for five animals that received live splenocytes are shown in Table 4, and representative histograms for mice that received live or irradiated splenocytes are presented in FIG. 2. The PBLs from NOD mice treated with irradiated CByB6F1 splenocytes showed only background staining for H-2K^{b}, indicating that no donor hematopoetic cells remain. In contrast, 4.4% to 12.6% of the PBLs from NOD mice treated with live CByB6F1 splenocytes were of donor origin. PBLs from an untreated NOD mouse contained only cells expressing H-2K^{d}, and those from a CByB6F1 mouse contained exclusively cells coexpressing H-2K^{b} and H-2K^{d}. NOD mice treated with live splenocytes thus exhibited a persistent low level of blood chimerism with semiallogeneic cells that was achieved without continuous immunosuppression or lethal preconditioning.

TABLE 4
Frequency and extent of donor engraftment in five NOD female mice with disease
reversal.

| | | | | | | Pancreatic ducts | | |
|---|---|---|---|---|---|---|---|---|
| | | Lymphoid system | | Islets | | Donor | | |
| NOD | | | Donor | Positive | Donor | pancreatic | Positive | Donor |
| Mouse | Age | Donor | splenocytes | for donor | composition | exocrine | for donor | composition |
| No. | (weeks) | PBLs (%) | (%) | cells (%) | (%) | cells (%) | cells (%) | (%) |
| #744 | 57 | 4.4 | 3.5 | 100 | 29 | 2 | 33 | 9 |
| #788 | 46 | 5.8 | 4.7 | 100 | 41 | 1 | 66 | 15 |
| #789 | 47 | 12.6 | 4.0 | 100 | 79 | 2 | 75 | 41 |
| #790 | 47 | 8.3 | 3.5 | 100 | 37 | 3 | 50 | 35 |
| #838 | 39 | 10 | 3.9 | 100 | 46 | 2 | 50 | 11 |
| Control | 38 | 0.3 | 0.3 | 3 | 2 | 2 | 1 | 1 |
| NOD | | | | | | | | |
| female | | | | | | | | |

### Example 4

### The Contribution of Exogenous Splenocytes to Islet Regeneration

The possibility that live injected CByB6F1 male splenocytes contribute to both diverse lymphoid cells and nonhematopoetic tissues, such as the newly appearing islets in the pancreas of successfully treated NOD females, was investigated. At 39 to 57 weeks of age, NOD mice with stable disease reversal induced by CFA and live CByB6F1 splenocytes were sacrificed for further analysis of hematopoetic and parenchymal chimerism.

Among the splenocytes from the five successfully treated NOD mice examined, flow cytometric analysis revealed the presence of from 3.5% to 4.7% of cells positive for both H-2K^{d} and H-2K^{b}, compared to a background level of 0.3% double-positive staining for splenocytes from an untreated control NOD mouse, confirming the persistence of donor CByB6F1 cells in all recipients. Differential gating for markers of various hematopoetic lineages revealed that CByB6F1 donor splenocytes contributed to T cells (CD3⁺), monocytes (Mac1⁺), and B cells (CD45R⁺).

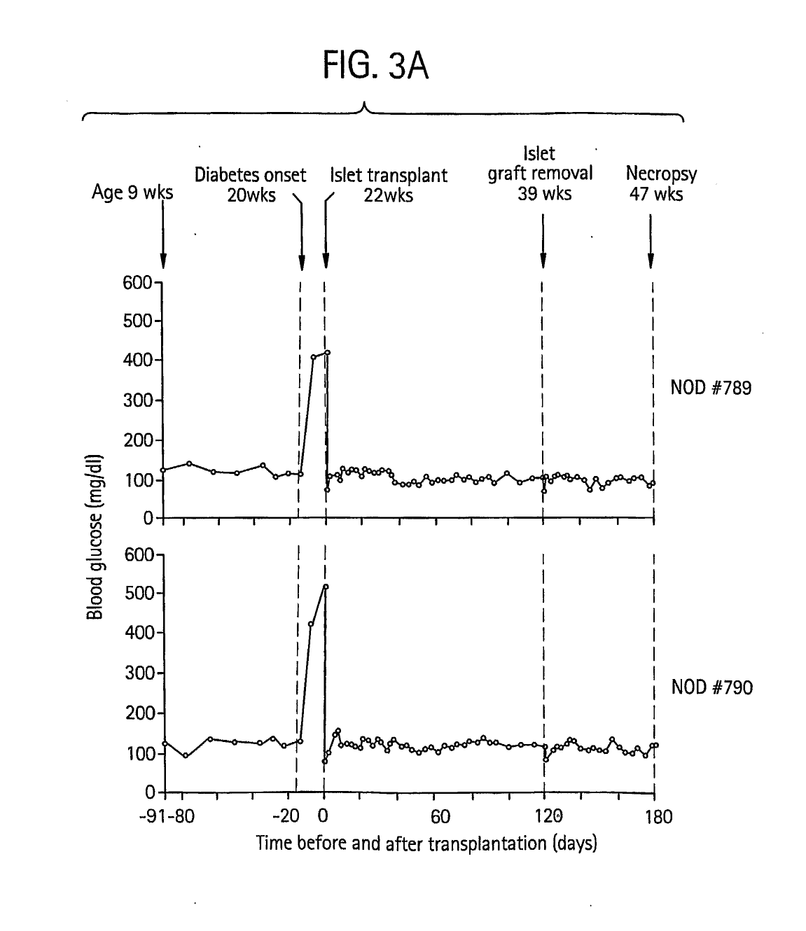

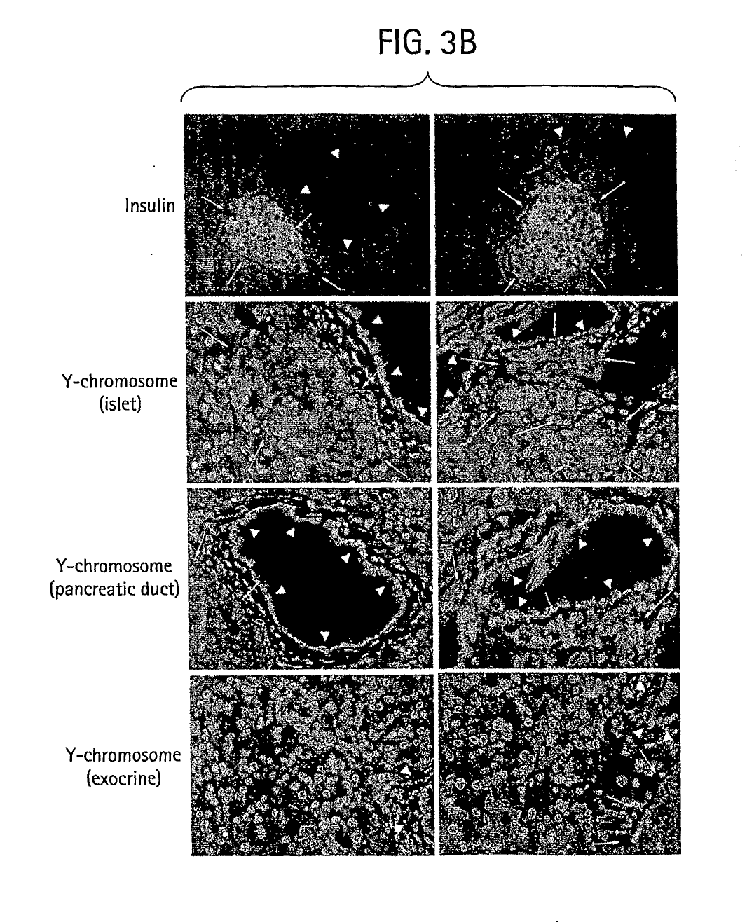

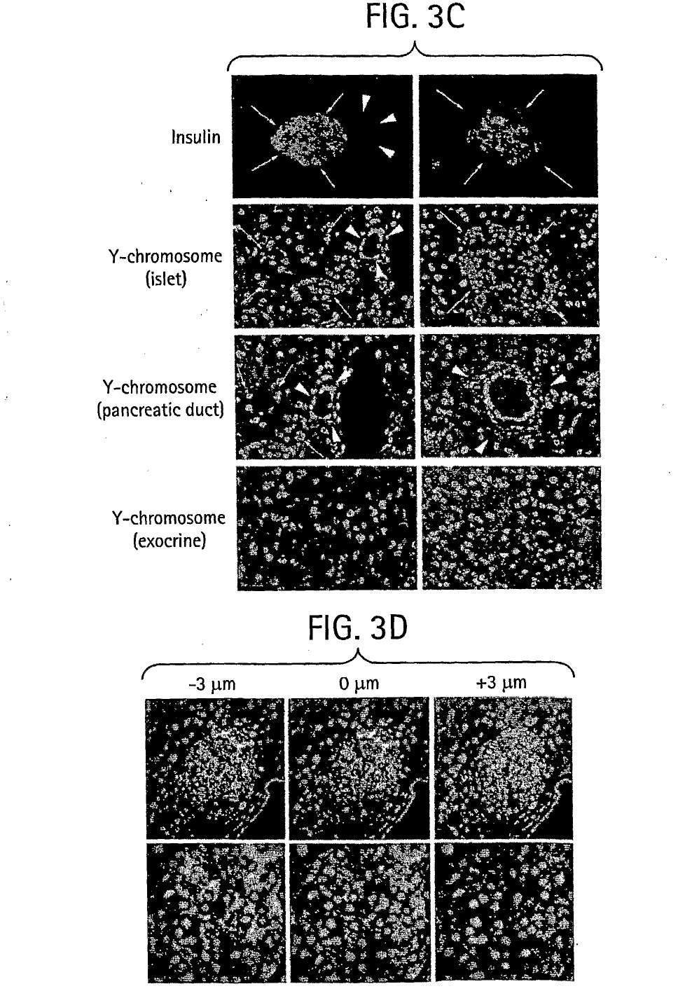

Parenchymal tissues were then examined for chimerism by FISH analysis for detection of the Y chromosome of the male donor cells. Serial sections of the pancreas were first evaluated for the presence of islets both by hematoxylin-eosin staining and by immunofluorescence analysis with antibodies to insulin and large well-formed islets were identified by both methods in the five successfully treated NOD mice examined. Data for two animals are shown in FIG. 3A and FIG. 3B. Blood glucose measurements demonstrated that therapy restored long-term normoglycemia until the mice were sacrificed at 47 weeks of age FIG. 3A. Staining of serial pancreatic sections with antibodies to insulin revealed a homogeneous insulin content in the large islets FIG. 3B, consistent with the restored normoglycemia. Single-color FISH analysis revealed the presence of abundant nuclei positive for the Y chromosome within the islets, as defined by morphology and insulin immunoreactivity FIG. 3B. In contrast, the exocrine portions of the pancreas were largely devoid of male cells. Similar results were obtained with all five treated NOD female mice examined. Quantitative analysis revealed that 29% to 79% of islet cells in these five animals were of donor origin. No islets solely of host origin were detected, consistent with the fact that the pancreas of NOD females before treatment lacks detectable islets as well as remaining clusters of insulitis.

Male donor cells also contributed to the epithelium of NOD female pancreatic ducts, although the distribution of male cells in this tissue was more heterogeneous than that found in the islets FIG. 3B. Among the five treated NOD females studied in detail, 33% to 75% of the ducts contained genetic material of male origin. Ducts purely of host origin were never associated with an adjacent islet containing male cells. The proportion of male cells in the pancreatic ducts of the five NOD mice ranged from 9% to 41%. Single-color FISH analysis revealed the presence of abundant nuclei positive for Y chromosomes within both the exocrine and endocrine portions of the pancreas of control C57BL/6 male mice, whereas the pancreas of control C57BL/6 females was devoid of Y chromosomes FIG. 3C.

The possibility that intrapancreatic lymphocytes were responsible for the Y chromosome signals detected in islets or pancreatic ducts of treated NOD females was excluded. As already shown, the introduction of live donor splenocytes uniformly eliminated invasive insulitis throughout the pancreas of NOD mice, as revealed by hematoxylin-eosin staining of complete sets of serial pancreatic sections; with lymphoid cells only rarely observed within the islets (FIG. 1C, Table 3). Furthermore, FISH analysis of tissue sections derived from the liver, brain, skin, or kidney of successfully treated NOD mice demonstrated the virtual absence of parenchymal signals for the Y chromosome, rendering it unlikely that normal intraparenchymal lymphoid cells, or passenger lymphocytes, were responsible for the Y chromosome signals in islets and pancreatic ducts.

Some recent studies have attributed the observed plasticity of adult stem cells in vivo to fusion with embryonic stem cells during prior culture (Terada et al., Nature 416: 542, 2002; Ying et al., Nature 416: 545, 2002). The hybrid cells contain markedly enlarged nuclei and multiple nucleoli and are tetraploid. With the use of serial sections and confocal microscopy, >800 nuclei in β cells as well as >800 nuclei in adjacent exocrine tissue of the five treated NOD females was studied in detail. Data for one of these animals are shown in FIG. 3D and Table 5. At three scanning focal lengths, none of the regenerated cells within the islets was enlarged compared with the adjacent native exocrine cells. The β-cell nuclei were of normal size and did not contain multiple nucleoli. These observations suggest that the regenerated islet cells were not the products of fusion between donor splenocytes and endogenous dying or injured β cells.

TABLE 5
Comparison of nuclear diameter between β cells and exocrine cells in a
successfully treated NOD mouse (#789 in Table 2).*

| | β cells | | Exocrine cells | | |
|---|---|---|---|---|---|
| | | Nuclear | | Nuclear | |
| Scanning | | diameter | | diameter | |
| position | Number | (pixels) | Number | (pixels) | P |
| −3 μm | 89 | 29.1 ± 4.0 | 91 | 36.0 ± 6.3 | 0.554 |
| 0 μm | 85 | 33.0 ± 4.3 | 91 | 36.8 ± 6.5 | 0.054 |
| (standard) | | | | | |
| +3 μm | 112 | 32.0 ± 5.8 | 102 | 33.8 ± 7.0 | 0.147 |

*Pancreatic sections stained with antibodies to insulin and propidium iodide were examined with a confocal microscope at three different focal planes. Nuclei in insulin-positive cells were counted as β-cell nuclei and those in insulin-negative cells in the exocrine portion of the pancreas were counted as exocrine cell nuclei. Nuclear diameter was determined by NIH Image software. Data are means ± SD for the indicated number of nuclei examined. The P values forcomparisons between islet and exocrine cells were obtained by Student's t test.

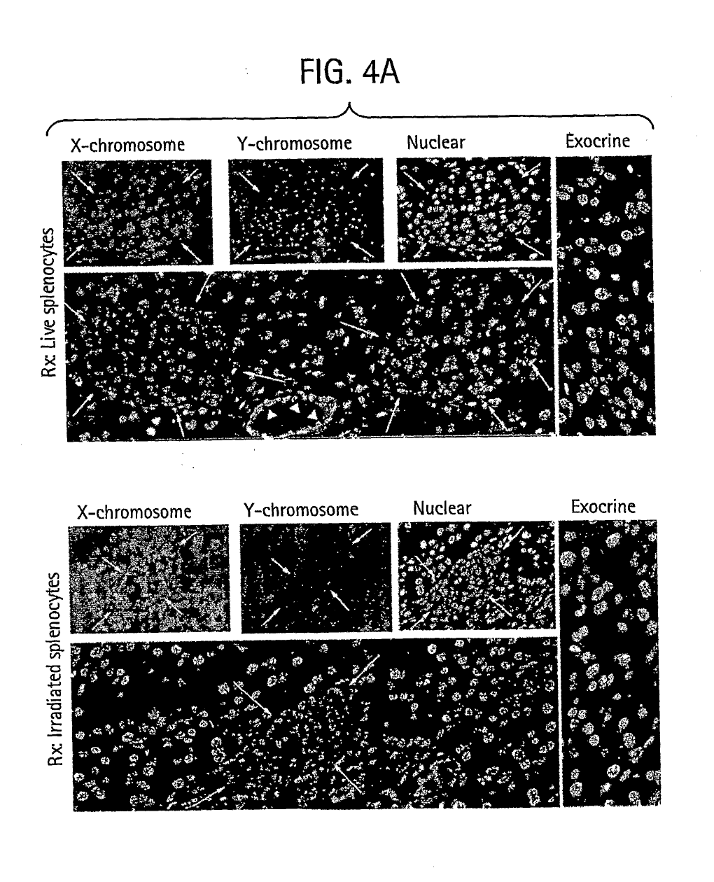

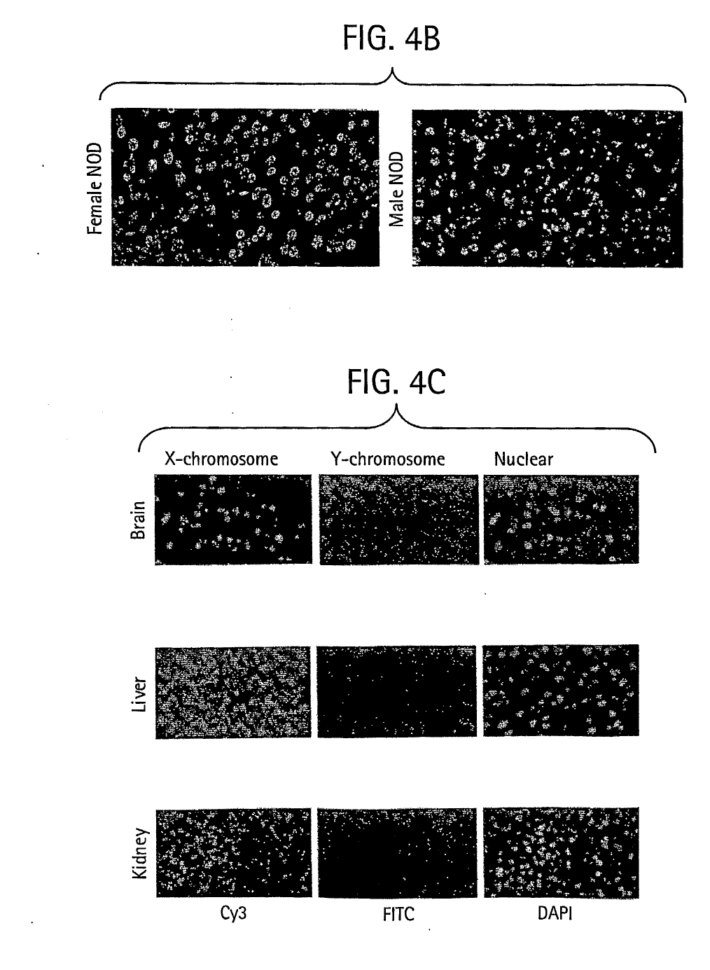

The ploidy of the sex chromosomes of cells in the regenerated islets of successfully treated NOD mice by two-color FISH analysis with a Y chromosome specific probe linked to fluorescein isothiocynate (FITC) (green) and an X chromosome-specific probe conjugated with Cyanine 3 (Cy3) (red) was further examined. Islet cell nuclei were also stained blue with 4′,6-diamidino-2-phenylindole (DAPI). FIG. 4A shows islets predominantly of male origin in a NOD female successfully treated with live splenocytes from CByB6F1 males. Inspection of individual nuclei revealed only rare if any islet cells with an apparent XXY or XXXY genotype. A normal complement of sex chromosomes was also observed in the pancreatic duct epithelium. These results thus again indicate that the regenerated islet cells were unlikely the result of frequent fusion between donor male cells and host female cells.

Similarly, the regenerated islets of a NOD female with long-term disease reversal due to treatment with CFA and irradiated splenocytes from CByB6F1 males were also examined. None of the islet cell nuclei contained a detectable Y chromosome, with each nucleus yielding two red signals, corresponding to a genotype of XX (FIG. 4A). Two-color FISH analysis of the pancreas of untreated female and male NOD mice revealed that, although this methodology can yield false negative data (female nuclei with no red signal or only one red signal), it almost never yielded false positive data (a green signal in the nucleus of a female cell or two green signals within an individual male nucleus) (FIG. 4B).

### Example 5

### The Analysis of Hox11 Expression

The spleens of adult C57BL/6, CByB6F1, NOD SCID, and NOD were examined by reverse transcription (RT) and polymerase chain reaction (PCR) analysis for Hox11 expression. The analyses revealed the presence of abundant Hox11 transcripts in the spleen of 12-well-old animals in each of the mouse strains examined (FIG. 5A). The presence of Hox11 mRNA in the spleen of NOD SCID mice, which lack most lymphoid cell populations, confirms that Hox11 is expressed in the nonlymphoid portions of the adult spleen. Pancreatic tissue from C57BL/6 embryos at embryonic day 15 (E15) did not contain Hox11 mRNA (FIG. 5A).

In addition, the spleens and pancreata of adult mice were examined for the expression of the Pdx1 gene, which marks the dorsal and ventral pancreatic buds between E8.5 and E16.5 (Offield et al., Development 122: 983-95, 1996). It was found that the spleens of adult mice do not contain Pdx1 mRNA, whereas the pancreata of C57BL/6 embryos at E15 do (FIG. 5A). Together, these data indicate that a pluripotent cell, the AGM cell, expresses Hox11, does not express the early pancreatic lineage marker Pdx1, and is not of lymphoid (CD45(+)) origin is present in the spleen of adult mice.

### Example 6

### The use of CD45(+) vs. CD45(−) Splenocytes

To examine the possible role of the AGM cell population in the regeneration of pancreatic islets in NOD mice treated with live donor splenocytes, 12-week-old NOD females (n=20) were injected with either CD45(+) or CD45(−) CByB6F1-GFP⁺ splenocytes, as well as unseparated splenocytes. All groups of NOD mice also received CFA and blood glucose and were monitored for >120 days. These experiments differ from previous experiments in that the NOD females used were prediabetic (i.e., with residual islet function but with active autoimmunity at the start of treatment), did not receive an islet graft, and the number of splenocytes cells they received by injection was reduced to 4×10⁵ to 5×10⁵, administered four times over 2 weeks. GFP fluorescence was use as a monitoring method to document re-growth of the islet cells from the injected donor splenocytes. All of the NOD females that received CD45(+) CByB6F1-GFP+(n=5) or CD45(−) (n=5) CByB6F1-GFP⁺, as well as those that received unseparated splenocytes (n=10) remained normoglycemic during the monitoring period, whereas all untreated NOD littermates (n=10) became diabetic under similar housing and observation conditions. The treated animals were subsequently sacrificed after 120 days of normoglycemia and the pancreata was subjected both to Western analysis for overall GFP+ expression (FIG. 5B) and serial pancreatic sections were subjected to immunohistochemical analysis for the detection of the individual fluorescence of CByB6F1-GFP+ cells in pancreatic islets (FIG. 5C). Sections were also stained with antibodies to CD45 and to insulin (FIG. 5D).

Long term normoglycemic NOD mice with successful disease reversal secondary to a brief low dose treatment with either CD45(+) or CD45(−) splenocytes from CByB6F1-GFP mice showed opposing GFP protein expression in the pancreas. Pancreatic extracts of NOD mice treated over 120 days earlier with enriched populations of CD45(−) splenocytes showed a strong GFP protein expression, with NOD mice treated with CD45(+) splenocytes having an almost undetectable GFP signal (FIG. 5B). Cytoplasmic pancreatic extracts of CByB6F1-GFP mice showed a strong band reactive with anti-GFP antibody, with control C57BL/6 mice showing no GFP reactive band (FIG. 5B). Co-treatment of prediabetic mice with CByB6F1 CD45(−) splenocyte and CFA resulted in the persistence of pancreatic located cell populations expressing a stable long-term GFP+ derivative of the introduced splenocyte population.

Similar to the results obtained in Example 5 with severely diabetic NOD mice treated with live splenocytes, the pancreata of prediabetic NOD females treated with either CD45(−) CByB6F1-GFP or unseparated CByB6F1-GFP spleen cells contained islets positive for the GFP marker (FIG. 5C). Furthermore, the newly generated islets lacked invasive lymphocytes and were associated with minimal or no peri-insulitis, as observed with insulin and CD45 co-staining (FIG. 5D). The number of islets of GFP origins appeared less frequent in prediabetic NOD females treated with CD45(−) or whole splenocytes than in severely diabetic NOD females, consistent with the fact that the pancreata of prediabetic mice still contained endogenous islets affected by peri-insulitis and that the treatment of pre-diabetic animals with mobilized precursor cells thus rescued damaged islets and also promoted de novo islet regeneration.

The pancreas of prediabetic NOD females treated with CD45(+) splenocytes also contained islets free of invasive insulitis. Immunohistochemical analysis, however, revealed the absence of islets positive for the GFP marker in these female hosts (FIG. 5C). Furthermore, similar to the islet regeneration observed in severely diabetic NOD mice treated with irradiated splenocytes in Example 5, the newly appearing islets in prediabetic NOD females treated with CD45(+) splenocytes exhibited pronounced peri-insulitis (FIG. 5C and FIG. 5D).

### Example 7

### Characterization of AGM Cells

### Isolation of AGM Cells

AGM cells, which are negative for CD45 expression, can be obtained from total spleen tissue mechanically teased apart with forceps and separated from CD45+ lymphoid cells (i.e., non-AGM cells) in the spleen using CD45-specific MicroBeads (Miltenyi Biotec, Auburn, Calif.). Furthermore all purifications and separations can be performed without the use of mesh sieves or the use of NH₄-tris-chloride, an agent useful for removing red blood cells from primary preparations. The red blood cell contamination can be eliminated by additional column purifications using Terr119 antibodies, a reagent specific for the red blood cell membrane. The procedures have been successfully utilized to isolated AGM cells in mice.

In mice spleens, AGM cells in normal animals are primarily confined to the capsular and subcapsular regions. In human spleens, the AGM cells are found concentrated in the capsule and subcapsular regions, but are also found throughout the pulp. AGM cells are also found in the human, although in less abundant amounts, in the tonsils/adenoids and thymus, thus, AGM cells of the invention can be purified from these sources as well. AGM cells in humans are not present in the bone marrow. AGM cells can also be purified using one or more of a number of cell surface markers expressed by AGM cells. These cell surface markers were identified by mass spectroscopy and are shown in the Tables 6 and 7. Miltenyi Biotec columns can be customized with purchased antibodies to these cell surface markers to purify the AGM cells of the invention to a higher degree. The isolation of AGM cells with or without additional purification can then be followed by in vitro or in vivo expansion, possibly using one or more of these cell surface markers as agonists to enhance cell expansion (e.g., a TNF receptor 1 agonist). AGM stimulating reagents known to expand these cells include the cytokines and hormones or agonists corresponding to the cell surface markers listed in Table 6. These cytokines can be used in vivo or in vitro. Further use of these cell surface markers can involve the use of agonist antibodies or agonist reagents to these cell surface markers or compounds working through these same pathways.

The mass spectrometry data also reveals that the AGM CD45− cells express a number of intracellular enzymes that allow the in vivo or in vitro stimulation of these cells. Furthermore, some of these reagents (cytokines or antibodies) when used in vitro can be used to further purify AGM cells. For example, a reagent can be used that selectively targets non-AGM CD45+ cells of the spleen but not the AGM cells due to expression, in the AGM cells, of one or more uniquely expressed enzymes that provide resistance to the reagent. For example, the AGM cells express the glyoxalase/bleomycin resistance protein/dihydroxybiphenyl dioxygenase. Expression of the protein allows the specific survival of AGM cells in the presence of bleomycin, while CD45+ cells, which do not express this protein, are susceptible to bleomycin and die in tissue culture. The addition of reagents or agonists of this pathway could also be used in vivo to expand the AGM cells during times of need. The mass spectrometry data also reveals the expression in the AGM cells of glycogen synthase kinase-3 beta (EC 2.7.1.37) (GSK-3 beta). GSK-3 participates in the Wnt signaling pathway, a pathway of development. GSK-3 is implicated in the hormonal control of several regulatory proteins including glycogen synthase, therefore drugs that inhibit this pathway could be used to release AGM cells from the spleen. Alternatively, drugs that stimulate GSK-3 could be used to expand the AGM cells in the spleen for future mobilization.

Similar methods can be applied to the other cell surface receptor identified in the AGM cells, such as, e.g., ADAM12. ADAM 12 induces actin cytoskeleton and extracellular matrix reorganization during early adipocyte and bone formation using the beta1 integrin pathway. As the mass spectrometry data shows, beta1 integrin is a receptor on AGM cells. The expression of ADAM12 in the AGM cells is believed to impair the function of beta1 integrin and, consequently, modulation of this receptor. Modulation of this receptor will not only have an effect on adipocyte or bone formation, but also for mesenchymal cell differentiation in general. Therefore, the hormones, through their receptors (i.e., the protein receptors listed in Table 6), could be utilized to facilitate the isolation or expansion of AGM cells. In addition, AGM cells can be screened using drugs that directly stimulate AGM cells due to the expression of a specific protein, according to the methodology described above.

TABLE 6
Cell Surface Membrane Receptors Identified on AGM Cells

| | | | Cell surface |
|---|---|---|---|
| | | | membrane |
| | | | receptors |
| Low-density lipoprotein | Potential cell surface | LRP4 | LDL receptor |
| receptor-related protein 4 | endocytic receptor, which | | |
| precursor (LDLR dan). | binds and internalizes | | |
| | extracellular ligands for | | |
| | degradation by lysosomes. | | |
| | SWISSPROT:LRP4_MOUSE | | |
| Ubiquilin 1 (Protein linking IAP | Links CD47 to the | UBQLN1 | CD47 |
| with cytoskeleton-1) (PLIC-1). | cytoskeleton. Promotes the | | |
| | surface expression of GABA- | | |
| | A receptors. Promotes the | | |
| | accumulation of uncleaved | | |
| | PSEN1 and PSEN2 by | | |
| | stimulating their biosynthesis. | | |
| | Has no effect on PSEN1 and | | |
| | PSEN2 degradation (By | | |
| | similarity). | | |
| | SWISSPROT:UBQ1_MOUSE | | |
| Mus musculus ES cells cDNA, | Unknown function | TRIP13 | thyroid hormone |
| RIKEN full-length enriched | | | receptor |
| library, clone: 2410002G23 | | | |
| product: thyroid hormone | | | |
| receptor interactor 13, full insert | | | |
| sequence. | | | |
| Mus musculus 6 days neonate | Unknown function | TREML1 | TLT-1 receptor |
| head cDNA, RIKEN full-length | | | |
| enriched library, | | | |
| clone: 5430401J17 | | | |
| product: hypothetical | | | |
| Immunoglobulin subtype | | | |
| containing protein, full insert | | | |
| sequence (Putative inhibitory | | | |
| receptor TLT-1). | | | |
| Mus musculus adult male | Unknown function | ITGA6 | Integrin receptor |
| diencephalon cDNA, RIKEN | | | alpha 6 |
| full-length enriched library, | | | |
| clone: 9330162A08 | | | |
| product: integrin alpha 6, full | | | |
| insert sequence. | | | |
| Mus musculus NOD-derived | Unknown function | LY78 | Surface receptor |
| CD11c +ve dendritic cells | | | CD78 |
| cDNA, RIKEN full-length | | | |
| enriched library, | | | |
| clone: F630107B15 | | | |
| product: lymphocyte antigen 78, | | | |
| full insert sequence. | | | |
| Aquaporin-CHIP (Water | Forms a water-specific | AQP1 | |
| channel protein for red blood | channel that provides the | | |
| cells and kidney proximal | plasma membranes of red | | |
| tubule) (Aquaporin 1) (Early | cells and kidney proximal | | |
| response protein DER2). | tubules with high permeability | | |
| | to water, thereby permitting | | |
| | water to move in the direction | | |
| | of an osmotic gradient. | | |
| | SWISSPROT:AQP1_MOUSE | | |
| Sodium/potassium-transporting | This is the catalytic | ATP1A4 | |
| ATPase alpha-4 chain (EC | component of the active | | |
| 3.6.3.9) (Sodium pump 4) | enzyme, which catalyzes the | | |
| (Na+/K+ ATPase 4). | hydrolysis of ATP coupled | | |
| | with the exchange of sodium | | |
| | and potassium ions across | | |
| | the plasma membrane. This | | |
| | action creates the | | |
| | electrochemical gradient of | | |
| | sodium and potassium ions, | | |
| | providing the energy for | | |
| | active transport of various | | |
| | nutrients. | | |
| | SWISSPROT:A1A4_MOUSE | | |
| Olfactory receptor MOR231-11 | Unknown function | OLFR1238 | olfactory |
| (Olfactory receptor Olfr1238). | | | receptor ie |
| | | | Olfr1238 |
| Olfactory receptor MOR239-4 | Unknown function | OLFR1427 | olfactory |
| (Olfactory receptor Olfr1427). | | | receptor ie |
| | | | Olfr1427 |
| Olfactory receptor MOR283-8 | Unknown function | OLFR693 | |
| (Olfactory receptor Olfr693). | | | |
| Caveolin-1. | May act as a scaffolding | CAV1 | |
| | protein within caveolar | | |
| | membranes. Interacts directly | | |
| | with G-protein alpha subunits | | |
| | and can functionally regulate | | |
| | their activity (By similarity). | | |
| | SWISSPROT:CAV1_MOUSE | | |
| T-cell surface glycoprotein CD3 | The CD3 complex mediates | CD3G | |
| gamma chain precursor (T-cell | signal transduction. | | |
| receptor T3 gamma chain). | SWISSPROT:CD3G_MOUSE | | |
| Coatomer beta subunit (Beta- | The coatomer is a cytosolic | COPB | |
| coat protein) (Beta-COP). | protein complex that binds to | | |
| | dilysine motifs and reversibly | | |
| | associates with Golgi non- | | |
| | clathrin-coated vesicles, | | |
| | which further mediate | | |
| | biosynthetic protein transport | | |
| | from the ER, via the Golgi up | | |
| | to the trans Golgi network. | | |
| | Coatomer complex is | | |
| | required for budding from | | |
| | Golgi membranes, and is | | |
| | essential for the retrograde | | |
| | Golgi-to-ER transport of | | |
| | dilysine-tagged proteins. In | | |
| | mammals, the coatomer can | | |
| | only be recruited by | | |
| | membranes associated to | | |
| | ADP-ribosylation factors | | |
| | (ARFs), which are small GTP- | | |
| | binding proteins; the complex | | |
| | also influences the Golgi | | |
| | structural integrity, as well as | | |
| | the processing, activity, and | | |
| | endocytic recycling of LDL | | |
| | receptors (By similarity). | | |
| | SWISSPROT:COPB_MOUSE | | |
| Glycophorin. | Glycophorin is the major | GYPA | |
| | intrinsic membrane | | |
| | sialoglycoprotein of the | | |
| | erythrocyte. | | |
| | SWISSPROT:GLP_MOUSE | | |
| Integrin alpha-2 precursor | Integrin alpha-2/beta-1 is a | ITGA2 | Find antibodies |
| (Platelet membrane | collagen receptor, being | | to CD49b |
| glycoprotein Ia) (GPIa) | responsible for adhesion of | | |
| (Collagen receptor) (VLA-2 | platelets and other cells to | | |
| alpha chain) (CD49b). | collagens, modulation of | | |
| | collagen and collagenase | | |
| | gene expression, force | | |
| | generation and organization | | |
| | of newly synthesized | | |
| | extracellular matrix. It is also | | |
| | a receptor for laminins, | | |
| | collagen C-propeptides and | | |
| | E-cadherin. Mice | | |
| | homozygous for a null | | |
| | mutation in the alpha-2 die | | |
| | very early in embryogenesis. | | |
| | SWISSPROT:ITA2_MOUSE | | |
| Integrin alpha-IIb precursor | Integrin alpha-IIb/beta-3 is a | ITGA2B | Cell surface |
| (Platelet membrane | receptor for fibronectin, | | receptor to CD41 |
| glycoprotein IIb) (GPalpha IIb) | fibrinogen, plasminogen, | | |
| (GPIIb) (CD41 antigen). | prothrombin, thrombospondin | | |
| | and vitronectin. It recognizes | | |
| | the sequence R-G-D in a | | |
| | wide array of ligands. It | | |
| | recognizes the sequence H- | | |
| | H-L-G-G-G-A-K-Q-A-G-D-V in | | |
| | fibrinogen gamma chain. | | |
| | Following activation integrin | | |
| | alpha-IIb/beta-3 brings about | | |
| | platelet/platelet interaction | | |
| | through binding of soluble | | |
| | fibrinogen. This step leads to | | |
| | rapid platelet aggregation | | |
| | which physically plugs | | |
| | ruptured endothelial cell | | |
| | surface. | | |
| | SWISSPROT:ITAB_MOUSE | | |
| Integrin alpha-6 precursor | Integrin alpha-6/beta-1 is a | ITGA6 | Find antibodies |
| (VLA-6) (CD49f). | receptor for laminin on | | to CD49f |
| | platelets. Integrin alpha- | | |
| | 6/beta-4 is a receptor for | | |
| | laminin in epithelial cells and | | |
| | it plays a critical structural | | |
| | role in the hemidesmosome. | | |
| | Mice expressing a null | | |
| | mutation of the alpha-6 | | |
| | subunit gene die soon after | | |
| | birth and develop severe | | |
| | blistering. The blisters are | | |
| | due to separation of the basal | | |
| | epithelial cells from a | | |
| | normally formed basement | | |
| | membrane. | | |
| | SWISSPROT:ITA6_MOUSE | | |
| Integrin alpha-M | Integrin alpha-M/beta-2 is | Itgam | Receptor for |
| | implicated in various | | iC3b |
| | adhesive interactions of | | |
| | monocytes, macrophages | | |
| | and granulocytes as well as | | |
| | in mediating the uptake of | | |
| | complement-coated particles. | | |
| | It is identical with CR-3, the | | |
| | receptor for the iC3b | | |
| | fragment of the third | | |
| | complement component. It | | |
| | probably recognizes the R-G- | | |
| | D peptide in C3b. Integrin | | |
| | alpha-M/beta-2 is also a | | |
| | receptor for fibrinogen, factor | | |
| | X and ICAM1. It recognizes | | |
| | P1 and P2 peptides of | | |
| | fibrinogen gamma chain. | | |
| | Alpha-M/beta-2 play a critical | | |
| | role in mast cell development | | |
| | and in immune complex- | | |
| | mediated glomerulonephritis. | | |
| | Mice expressing a null | | |
| | mutation of the alpha-M | | |
| | subunit gene demonstrate | | |
| | increase in neutrophil | | |
| | accumulation, in response to | | |
| | a impaired degranulation and | | |
| | phagocytosis, events that | | |
| | apparently accelerate | | |
| | apoptosis in neutrophils. | | |
| | These mice develop obesity. | | |
| Integrin alpha-V precursor | The alpha-V integrins are | ITGAV | Find antiboides |
| (Vitronectin receptor alpha | receptors for vitronectin, | | to CD51 |
| subunit) (CD51 antigen). | cytotactin, fibronectin, | | |
| | fibrinogen, laminin, matrix | | |
| | metalloproteinase-2, | | |
| | osteopontin, osteomodulin, | | |
| | prothrombin, thrombospondin | | |
| | and von Willebrand factor. | | |
| | They recognize the sequence | | |
| | R-G-D in a wide array of | | |
| | ligands. Alpha-V integrins | | |
| | may play a role in embryo | | |
| | implantation, angiogenesis | | |
| | and wound healing. Mice | | |
| | expressing a null mutation of | | |
| | the alpha-V subunit gene | | |
| | survive until late in embryonic | | |
| | development and | | |
| | occasionally even to birth. | | |
| | They demonstrate cleft | | |
| | palate, and defective | | |
| | development of CNS and | | |
| | gastrointestinal blood | | |
| | vessels. | | |
| | SWISSPROT:ITAV_MOUSE | | |
| Integrin beta-3 precursor | Integrin alpha-V/beta-3 is a | ITGB3 | Recptors to |
| (Platelet membrane | receptor for cytotactin, | | CD61 |
| glycoprotein IIIa) (GPIIIa) | fibronectin, laminin, matrix | | |
| (CD61 antigen). | metalloproteinase-2, | | |
| | osteopontin, osteomodulin, | | |
| | prothrombin, | | |
| | thrombospondin, vitronectin | | |
| | and von Willebrand factor. | | |
| | Integrin alpha-IIB/beta-3 is a | | |
| | receptor for fibronectin, | | |
| | fibrinogen, plasminogen, | | |
| | prothrombin, thrombospondin | | |
| | and vitronectin. Integrins | | |
| | alpha-IIB/beta-3 and alpha- | | |
| | V/beta-3 recognize the | | |
| | sequence R-G-D in a wide | | |
| | array of ligands. Integrin | | |
| | alpha-IIB/beta-3 recognizes | | |
| | the sequence H-H-L-G-G-G- | | |
| | A-K-Q-A-G-D-V in fibrinogen | | |
| | gamma chain. Following | | |
| | activation integrin alpha- | | |
| | IIB/beta-3 brings about | | |
| | platelet/platelet interaction | | |
| | through binding of soluble | | |
| | fibrinogen. This step leads to | | |
| | rapid platelet aggregation | | |
| | which physically plugs | | |
| | ruptured endothelial surface. | | |
| | SWISSPROT:ITB3_MOUSE | | |
| Laminin beta-3 chain precursor | Binding to cells via a high | LAMB3 | Recpeto of |
| (Laminin 5 beta 3) (Kalinin B1 | affinity receptor, laminin is | | laminin receptor |
| chain). | thought to mediate the | | |
| | attachment, migration and | | |
| | organization of cells into | | |
| | tissues during embryonic | | |
| | development by interacting | | |
| | with other extracellular matrix | | |
| | components. | | |
| | SWISSPROT:LMB3_MOUSE | | |
| Neurogenic locus notch | Functions as a receptor for | NOTCH3 | Cell surface |
| homolog protein 3 precursor | membrane-bound ligands | | receptor: Notch 3 |
| (Notch 3). | Jagged1, Jagged2 and | | receptor |
| | Delta1 to regulate cell-fate | | |
| | determination. Upon ligand | | |
| | activation through the | | |
| | released notch intracellular | | |
| | domain (NICD) it forms a | | |
| | transcriptional activator | | |
| | complex with RBP-J kappa | | |
| | and activates genes of the | | |
| | enhancer of split locus. | | |
| | Affects the implementation of | | |
| | differentiation, proliferation | | |
| | and apoptotic programs (By | | |
| | similarity). May play a role | | |
| | during CNS development; | | |
| | Constitutively active | | |
| | expression Notch3-induced | | |
| | tumors-primarily as splenic | | |
| | lymphomas. | | |
| | SWISSPROT:NTC3_MOUSE | | |
| Pactolus. | Cell surface protein | ITGB2L | |
| | preferentially expressed by | | |
| | neutrophils | | |
| Patched protein homolog 1 | Acts as a receptor for sonic | PTCH | Receptor: |
| (PTC1) (PTC). | hedgehog (SHH), indian | | patched protein |
| | hedgehog (IHH) and desert | | homolog 1 |
| | hedgehog (DHH). Associates | | (PTC1) |
| | with the smoothened protein | | |
| | (SMO) to transduce the | | |
| | hedgehog's proteins signal. | | |
| | Seems to have a tumor | | |
| | suppressor function, as | | |
| | inactivation of this protein is | | |
| | probably a necessary, if not | | |
| | sufficient step for | | |
| | tumorigenesis. | | |
| | SWISSPROT:PTC1_MOUSE | | |
| Mus musculus 0 day neonate | EA3; Type I membrane | none | Cell surface |
| cerebellum cDNA, RIKEN full- | protein (By similarity), | | protein: integrin |
| length enriched library, | | | alpha 2b |
| clone: C230009P15 | | | |
| Product: integrin alpha 2b, full | | | |
| insert sequence. | | | |
| Mus musculus adult male | Involved with interleukin- | ITGA6 | Recptor or |
| diencephalon cDNA, RIKEN | 1alpha to increase metastatic | | protein IL1a |
| full-length enriched library, | capability of human | | |
| clone: 9330162A08 | pancreatic cancer; Interacts | | |
| product: integrin alpha 6, full | with Shc and Grb2 in the | | |
| insert sequence. | differentiating cortical fiber | | |
| CaV2.1. | Unknown function | CACNA1A | |
| C lectin-related protein B | Inhibitory lectin that inhibits | OCIL | |
| (Fragment). | the formation and function of | | |
| | osteoclasts | | |
| C-type lectin-like receptor 2. | Unknown function | CLEC2 | |
| Syntenin 1 (Syndecan binding | Seems to function as an | SDCBP | Recptor or ligand |
| protein 1) (Scaffold protein | adapter protein. In adherens | | to IL5 receptor |
| Pbp1). | junctions may function to | | |
| | couple syndecans to | | |
| | cytoskeletal proteins or | | |
| | signaling components. | | |
| | Seems to couple transcription | | |
| | factor SOX4 to the IL-5 | | |
| | receptor (IL5RA). May also | | |
| | play a role in vesicular | | |
| | trafficking. Seems to be | | |
| | required for the targeting of | | |
| | TGFA to the cell surface in | | |
| | the early secretory pathway. | | |
| | SWISSPROT:SDB1_MOUSE | | |
| Toll-interacting protein. | Component of the signaling | TOLLIP | |
| | pathway of IL-1 and Toll-like | | |
| | receptors. Inhibits cell | | |
| | activation by microbial | | |
| | products. Recruits IRAK1 to | | |
| | the IL-1 receptor complex. | | |
| | Inhibits IRAK1 | | |
| | phosphorylation and kinase | | |
| | activity. | | |
| | SWISSPROT:TLIP_MOUSE | | |
| Tumor necrosis factor receptor | Receptor for TNFSF2/TNF- | TNFRSF1A | Soluable TNF or |
| superfamily member 1A | alpha and homotrimeric | | TNF stimulation |
| precursor (p60) (TNF-R1) (TNF- | TNFSF1/lymphotoxin-alpha. | | or specific |
| RI) (p55). | The adaptor molecule FADD | | agonist to |
| | recruits caspase-8 to the | | TNFR1 |
| | activated receptor. The | | |
| | resulting death-inducing | | |
| | signaling complex (DISC) | | |
| | performs caspase-8 | | |
| | proteolytic activation which | | |
| | initiates the subsequent | | |
| | cascade of caspases | | |
| | (aspartate-specific cysteine | | |
| | proteases) mediating | | |
| | apoptosis (By similarity). | | |
| | SWISSPROT:TR1A_MOUSE | | |
| Vesicule-associated membrane | May participate in trafficking | VAMP5 | |
| protein 5 (VAMP-5) | events that are associated | | |
| (Myobrevin). | with myogenesis, such as | | |
| | myoblast fusion and/or | | |
| | GLUT4 trafficking. | | |
| | SWISSPROT:VAM5_MOUSE | | |
| CD97 antigen precursor. | Receptor potentially involved | CD97 | Cell surface |
| | in both adhesion and | | receptor to CD97 |
| | signaling processes early | | |
| | after leukocyte activation. | | |
| | Plays an essential role in | | |
| | leukocyte migration. | | |
| | SWISSPROT:CD97_MOUSE | | |

### Fractionation of AGM Cells

The separation of CD45(+) and CD45(−) spleen cells from normal B6 donor mice was achieved by using mouse-specific CD45 MicroBeads (Miltenyi Biotec, Auburn, Calif.) as described above for CByB6F1 donor mice. Each of the CD45(+) and CD45(−) spleen cell populations was fractionated into a nuclear, a cytoplasmic and a membrane fraction and then further fractionated by SDS PAGE. The gel lanes were divided horizontally into 10 slices and the contents of each horizontal division subjected to an in-gel trypsin digest. Each of the resulting peptide mixtures was analyzed in an automated system by nano-scale LC-MS/MS over 24 hours as described below.

### Characterization of Cells by Mass Spectrometry

Mass spectroscopy data obtained from CD45− AGM cells revealed developmental proteins indicative of the persistence of a fetal stem cell in an adult animal. The protein profiling showed developmental specific proteins that control the formation of the fetal nervous system (cerebellum, neurogenesis, axon guidance), blood vessels/heart/skeletal muscle/tongue, pancreas, skin, olfactory system cerebellum, dorsal root ganglia/cranial nerves, eye and ear development, proteins involved in exocrine and endocrine development that interact with Hox11 (pax4, 5, 6) and gonads (gametogenesis, spermatogenesis). A partial list of these proteins is provided in Table 7. Additional proteins in the Wnt, Hedgehog and Notch family confirmed the primitive origins of these AGM cells. Although some of these proteins have been specifically detected in day 0 fetal development, the majority of the fetal proteins are abundantly apparent in day 10-11 of gestation). Therefore this analysis of the CD45− fractions of the spleen is consistent with a stem cell population that represents a frozen fetal cluster of a mid-stage murine embryo and of an AGM lineage. Table 8 provides a partial list of tissue specific proteins expressed by AGM cells, which can be used to predict the lineage that these cells are capable of differentiating into upon implantation or in vivo stimulation.

TABLE 7
Developmental and Adult Organ Specific Proteins Expressed by AGM
Cells

| MS/MS ID | Accession | Gene | |
|---|---|---|---|
| | | | Developmental Protein function |
| XRN2_MOUSE | Q9DBR1 | XRN2 | Developmental protein invovlved in |
| | | | gametogenesis and spermatogenesis; |
| GDN_MOUSE | Q07235 | SERPINE2 | Glia derived nexin precursor (GDN) |
| | | | (Protease nexin I) (PN-1) (Serine |
| | | | protease-inhibitor-4). |
| AK017688 | Q9CYH2 | 5730469M10RIK | Mus musculus 8 days embryo whole |
| | | | body cDNA, |
| AK020751 | Q9D211 | D11ERTD497E | Mus musculus 0 day neonate thymus |
| | | | cDNA, |
| AK048780 | Q8BX68 | HDLBP | Mus musculus 0 day neonate |
| | | | cerebellum cDNA, |
| AK028842 | Q8C174 | 5730596K20RIK | Mus musculus 10 days neonate skin |
| | | | cDNA, |
| K2CA_HUMAN | P02538 | KRT6A | Developmental protein invovled in |
| | | | ectoderm development; Keratin, type II |
| | | | cytoskeletal 6A (Cytokeratin 6A) (CK |
| | | | 6A) (K6a keratin). |
| AK088094 | Q8BU13 | 2010001M09RIK | Mus musculus 2 days neonate thymus |
| | | | thymic cells cDNA, |
| AK075879 | Q922H2 | PDK3 | (Mus musculus 10 days embryo whole |
| | | | body cDNA, |
| PLF4_MOUSE | Q9Z126 | PF4 | Developmental protein involved in blood |
| | | | vessel development and angiogenesis |
| | | | as well as fetal karyocyte differentiation; |
| AK013263 | Q9CYV8 | 1500019G21RIK | Mus musculus 10, 11 days embryo |
| | | | whole body cDNA, |
| AK014623 | Q9D651 | 4633402N23RIK | Mus musculus 0 day neonate skin |
| | | | cDNA, |
| AK076000 | Q8BK74 | SCAP2 | Mus musculus 10 days embryo whole |
| | | | body cDNA, |
| KF5C_MOUSE | P28738 | KIF5C | Developmental protein involved in |
| | | | neuogenesis, axon guidance and |
| | | | aconogenesis; Kinesin heavy chain |
| | | | isoform 5C (Kinesin heavy chain |
| | | | neuron-specific 2). |
| PTNB_MOUSE | P35235 | PTPN11 | Developmental protein invovled in |
| | | | neurogenesis and acongeogenesis; |
| | | | Protein-tyrosine phosphatase, non- |
| | | | receptor type 11 (EC 3.1.3.48) (Protein- |
| | | | tyrosine phosphatase SYP). |
| AK004472 | Q9D0T8 | 1190003K14RIK | Mus musculus 18-day embryo whole |
| | | | body cDNA, |
| AK007710 | Q9D8T2 | GSDMDC1 | Mus musculus 10 day old male |
| | | | pancreas cDNA, |
| AK011914 | Q9D014 | 2610207P08RIK | Mus musculus 10 days embryo whole |
| | | | body cDNA, |
| AK012340 | Q9CZP2 | 1110025F24RIK | Mus musculus 11 days embryo whole |
| | | | body cDNA, |
| | | | Adult Organ Specific Function |
| AK018758 | Q9D2V2 | 1300006C19RIK | Mus musculus adult male liver cDNA, |
| AK004764 | Q9DBT1 | F13A | Mus musculus adult male lung cDNA, |
| AK075879 | Q922H2 | PDK3 | Mus musculus adult male tongue cDNA, |
| AK013460 | Q9D6K8 | 4833415N24RIK | Mus musculus adult male hippocampus |
| | | | cDNA, |
| AK018652 | Q9D2X9 | D19ERTD703E | Mus musculus adult male cecum cDNA, |
| AK002843 | Q9DCE9 | IGTP | Mus musculus adult male kidney cDNA, |

TABLE 8
AGM Cell Tissue Specific Proteins Related to Differentiation Potential

| MS/MS ID | Accession | Gene | |
|---|---|---|---|
| | | | Developmental Protein function |
| R23B_MOUSE | P54728 | RAD23B | Developmental protein involved with |
| | | | reproduction, gametogenesis, male |
| | | | gamete generation and |
| | | | spermatogenesis. |
| AK051230 | Q922Q1 | 2810484M10RIK | RIKEN cDNA 2810484M10 (Mus |
| | | | musculus 12 days embryo spinal |
| | | | ganglion cDNA, |
| AK077912 | Q8BVK3 | 5730445F03RIK | Mus musculus 13 days embryo male |
| | | | testis cDNA, |
| AK034609 | Q99JT2 | 2610018G03RIK | Mst3 and SOK1-related kinase (Mus |
| | | | musculus 12 days embryo embryonic |
| | | | body between diaphragm region and |
| | | | neck cDNA, |
| AK004535 | Q9JHW2 | D16ERTD502E | Nit protein 2 (Mus musculus 18-day |
| | | | embryo whole body |
| KAPA_MOUSE | P05132 | PRKACA | Developmental protein involved with |
| | | | fetal development, morphogenesis, |
| | | | organogenesis, gastrulation, |
| | | | histogenesis, formation of primary germ |
| | | | layers, and mesoderm development. |
| | | | cAMP-dependent protein kinase, alpha- |
| | | | catalytic subunit (EC 2.7.1.37) (PKA C- |
| | | | alpha). |
| AK003076 | Q9CQ43 | DUTP | Mus musculus 10 days embryo whole |
| | | | body |
| AK003463 | Q9D1J1 | 1110005F07RIK | Mus musculus 18-day embryo whole |
| | | | body cDNA, |
| AK003604 | Q9CTG9 | Nit2; | Mus musculus 18-day embryo whole |
| | | D16ERTD502E | body cDNA, |
| AK004208 | Q8BMZ1 | 1110049F12RIK | Mus musculus 18-day embryo whole |
| | | | body cDNA, |
| AK011525 | Q9D0D9 | NT5C3 | Mus musculus 10 days embryo whole |
| | | | body cDNA, |
| | | | Adult Organ Specific Function |
| AK004919 | Q9CW46 | 1300006N24RIK | Mus musculus adult male liver cDNA, |

Identification and sequence analysis of peptide mixtures were performed in an automated system by nano-scale micro capillary LC-MS/MS on a Finnigan™ LCQ™ Deca XP (thermo electron) ion trap mass spectrometer, consisting of an autosampler, a capillary HPLC system connected to an autosampler, an ion trap MS, and a data system. In-gel digestion samples were individually placed into an autosampler vial. The samples were subsequently automatically loaded onto a 75 μm i.d. column, which is first washed at 5 μl/min with 0.4% acetic acid/0.005% heptafluorobutyric acid/5% acetonitrite/95% water and then eluted at ˜150 nl/min with a gradient system in which the acetonitrile concentration was incrementally increased. The mass spectrometer was operated in dual mode for measuring m/z ratios for eluting peptides (MS mode) and collecting sequencing information for eluting peptide ions whose intensity reaches a certain threshold (MS/MS) mode. Approximately 3000-4000 MS/MS spectra were acquired during a typical two-hour gradient for each sample, with each MS/MS spectrum containing sequence information for only one peptide.

Using the MS described above protein differences were identified for CD45(−) vs. CD45(+) spleen cells harvested from normal mice. A partial listing of proteins characteristic of an adult CD45(−)/Hox11-expressing AGM cell of the present invention is shown in Table 6 (above) and Table 9.

TABLE 9
Proteins characteristic of adult CD45(−)/Hox11-expressing splenocytes

| | | | Tissue or membrane | |
|---|---|---|---|---|
| ID | ACC | Name | specificity to the protein | GENE |
| A32E_MOUSE | P97822; | Acidic leucine-rich | Controls the | ANP32E; |
| | Q8BPF8; | nuclear phosphoprotein 32 | development of the | CPD1 |
| | Q8C2L4; | family member E; | cerebellum | |
| | Q8C7Q8; | Cerebellar postnatal | | |
| | Q9CZD2 | development protein-1; | | |
| | | LANP-L; LANP-like | | |
| | | protein | | |
| BCA1_MOUSE | Q61140; | CRK-associated substrate; | Associated with a | BCAR1; |
| | Q60869 | Breast cancer anti- | membrane estrogen | CRKAS; CAS |
| | | estrogen resistance 1 | receptor for cellular | |
| | | protein; p130cas | identification, | |
| | | | purification or | |
| | | | stimulation of growth | |
| COE2_MOUSE | O08792 | Transcription factor | Obligatory for olfactory | EBF2; COE2; |
| | | COE2; Early B-cell factor | development, the | MMOT1 |
| | | 2; OE-3; O/E-3; | metencephalon- | |
| | | Metencephalon- | mesencephalon | |
| | | mesencephalon-olfactory | development in the | |
| | | transcription factor 1; | brain. | |
| | | MET-mesencephalon- | | |
| | | olfactory TF1; MET- | | |
| | | mesencephalon-olfactory | | |
| | | transcription factor 1; | | |
| | | EBF-2; Olf-1/EBF-like 3 | | |
| DRG1_MOUSE | P32233 | Developmentally | Fetal development | DRG1; |
| | | regulated GTP-binding | protein | NEDD3; |
| | | protein 1; Nedd3 protein; | | NEDD-3; |
| | | DRG 1 | | DRG |
| DRG2_MOUSE | Q9QXB9 | Developmentally | Fetal development | DRG2 |
| | | regulated GTP-binding | protein | |
| | | protein 2; DRG 2 | | |
| EYA3_MOUSE | P97480; | Eyes absent homolog 3 | Obligatory for the | EYA3 |
| | P97768 | | development of eye, | |
| | | | cranium, brachial arches, | |
| GOA3_MOUSE | P55937; | Golgi autoantigen, golgin | | GOLGA3; |
| | Q80VF5; | subfamily A member 3; | | MEA2 |
| | Q8CCK4; | Golgin-160; MEA-2; | | |
| | Q9QYT2; | Male-enhanced antigen-2 | | |
| | Q9QYT3 | | | |
| GX42_MOUSE | Q91XR9; | Phospholipid | Obligatory for | GPX4 |
| | Q8K4U8 | hydroperoxide glutathione | embryonic development. | |
| | | peroxidase, nuclear; GPX-4 | | |
| HORN_MOUSE | Q8VHD8 | Hornerin | Developmental protein | |
| | | | obligatory for | |
| | | | keratinization - skin | |
| | | | development | |
| PAX5_MOUSE | Q02650 | Paired box protein Pax-5; | Transcription factor for | PAX5; PAX-5 |
| | | B-cell specific | spermatogenesis, | |
| | | transcription factor; BSAP | neurogenesis. Interacts | |
| | | | with Hox11 in the brain | |
| | | | at the pallio-subpallial | |
| | | | boundary. Obligatory for | |
| | | | the both exocrine and | |
| | | | endocrine development | |
| | | | of pancreas, islets, | |
| | | | salivary glands, | |
| | | | pituitary, adrenals, etc. | |
| PICA_MOUSE | Q7M6Y3; | Phosphatidylinositol- | | PICALM; |
| | Q811P1; | binding clathrin assembly | | CALM; FIT1 |
| | Q8BUF6; | protein; CALM; Clathrin | | |
| | Q8CIH8; | assembly lymphoid | | |
| | Q8R0A9; | myeloid leukemia | | |
| | Q8R3E1; | | | |
| | Q8VDN5; | | | |
| | Q921L0 | | | |
| RELN_MOUSE | Q60841; | Reelin precursor; Reeler | Developmental protein | RELN; RL |
| | Q9CUA6 | protein | and a protein expressed | |
| | | | on the cell as EGF like | |
| | | | domains This protein | |
| | | | can also be expressed in | |
| | | | the normal adult brain | |
| SLI1_MOUSE | P97447; | Skeletal muscle LIM- | Controls skeletal and | FHL1 |
| | O55181; | protein 1; SLIM 1; | heart development | |
| | Q8K318 | RAM14-1; Four and a half | | |
| | | LIM domains protein 1; | | |
| | | RBP associated molecule | | |
| | | 14-1; FHL-1; KyoT | | |
| SORL_MOUSE | O88307; | Sortilin-related receptor | Developmental protein | SORL1 |
| | O54711; | precursor; LDLR relative | that has EGF exterior | |
| | O70581 | with 11 ligand-binding | surface domains which | |
| | | repeats; mSorLA; LR11; | may allow for the | |
| | | Low-density lipoprotein | identification of cells by | |
| | | receptor relative with 11 | this cell surface marker | |
| | | ligand-binding repeats; | determinant. This | |
| | | Gp250; SorLA-1; Sorting | protein controls brain | |
| | | protein-related receptor | development, cerebral | |
| | | containing LDLR class A | development and spleen | |
| | | repeats | development | |
| WNT2_MOUSE | P21552 | Wnt-2 protein precursor; | Stem cell marker and | WNT2; WNT- |
| | | INT-1 related protein; IRP | part of the Wnt signaling | 2; IRP |
| | | protein | pathway. | |
| | | Syntaxin 4 | This is an adult protein | |
| | | | but previously only | |
| | | | found in synaptic | |
| | | | vesicles in neurons | |
| | RB3D | Ras related protein Rab-#d | Possible involvement in | |
| | | | insulin induced | |
| | | | exocytosis of glut4 | |
| | | | containing vesicles in | |
| | | | adipocytes | |
| | GDN | Glia derived nexin | Promotes neurite | |
| | | precursor | extension | |
| | ADRO | NADPH | Involved with adrenal | |
| | | | gland development | |
| | IF32 | TGF beta receptor | Cell surface marker for | |
| | | interacting protein | TGF protein. | |
| | RNP2 | RNA binding region | Expressed on the cell | |
| | | containing protein 2; | surface | |
| | | coactivator of achieving | | |
| | | protein 1 and estrogen | | |
| | | receptors | | |
| | RUV1 | RuvB-like1 | A plasminogen receptor | |
| | GPX-4 | Phospholipid | Essential for embryonic | |
| | | hydroperoxide glutathione | development | |
| | | peroxidase | | |
| | T30827 | Nascent polypeptide | Developmental protein | |
| | | associated complex | for muscle formation | |
| | ASC1 | Achaete scute homolog 1, | A developmental protein | |
| | | Mash1, Ash1 | of specific neural | |
| | | | lineages in most regions | |
| | | | of the CNS and of the | |
| | | | PNS. Essential for the | |
| | | | generation of olfactory | |
| | | | and autonomic neurons. | |
| | | | Forms a herodimer with | |
| | | | bHLH protein. | |
| | | | Expressed between | |
| | | | embryonic days 8.5-10.5 | |
| | | | and is found in the | |
| | | | neuroepithelium of the | |
| | | | midbrain and ventral | |
| | | | forebrain as well as in | |
| | | | the spinal cord. | |
| | | | Between days 10.5 and | |
| | | | 12.5 its expression | |
| | | | pattern changes from a | |
| | | | restricted to a | |
| | | | widespread one and it is | |
| | | | then found at the | |
| | | | ventricular zone of all | |
| | | | regions of the brain. | |
| | | | From day 12.5 to birth it | |
| | | | is expressed in the | |
| | | | ventricular zone through | |
| | | | the brain and the | |
| | | | olfactory epithelium and | |
| | | | retina. | |
| | ITA2 | LA-2, Integrin alpha 2 | Expressed on the cell | |
| | | precursor, CD49B | surface | |
| | ITAB | CD41 | Expressed on the cell | |
| | | | surface | |
| | ITAL | LFA-1 | Expressed on the cell | |
| | | | surface | |
| | ITB2 | | Developmental protein. | |
| | | | A cell surface adhesion | |
| | | | lipoprotein LFA-1 | |
| | | | integrin beta 2 precursor | |
| | | | Expressed on the cell | |
| | | | surface. | |
| | ITB1 | CD29 | Expressed on the cell | |
| | | | surface | |
| | ITB5 | Itgb5 | Developmental protein, | |
| | | | (intefrin beta 5) | |
| | | | important for cell growth | |
| | | | and expansion | |
| | EZR1 | Cytovillin, Ezrin | Cytoplasm | |
| | | | developmental protein | |
| | | | involved in the | |
| | | | formation of micovilli of | |
| | | | intestinal epithelial cells. | |
| | NTC3 | Neurogenic locus notch | A Notch developmental | |
| | | homoglo protein 3 | protein expressed in the | |
| | | | neruoepithelium. | |
| | | | Important for presenilin | |
| | | | proteolysis. This protein | |
| | | | also affects the | |
| | | | implementation of | |
| | | | differenation, | |
| | | | proliferation for CNS | |
| | | | development. Binds to | |
| | | | the following ligands to | |
| | | | regulate cell fate | |
| | | | determination: Jagged1, | |
| | | | Jagged2, and Delta1. | |
| | EHD1 | EH domain containing | Expressed in 9.5 day | |
| | | protein 1; PAST1 | embryo in the brachial | |
| | | | apparatus (mandible and | |
| | | | hyoid) and in the various | |
| | | | elements of the | |
| | | | pharyngeal arches at at | |
| | | | day 10.5 in scleroses. IN | |
| | | | adults this protein can be | |
| | | | found in kidney, heart, | |
| | | | intestine and brain | |
| | TFR1 | Transferrin receptor | Transferrin receptor | |
| | | protein 1 | protein 1 | |
| | AK031357 | Adrenal Gland | Controls the | |
| | | | development of the | |
| | | | adrenal gland | |
| | AD088149 | Hemoglobin, beta adult | Only expressed in day | |
| | | | 11 embryo | |
| | BC062899 | Acid nuclear phospho | Only expressed in | |
| | | | cerebellar tissue | |
| | SYUA | Alpha synuclein, Amyloid | Commonly expressed in | |
| | | | adult brain and regulated | |
| | | | dopamine release and | |
| | | | transport | |
| | CAD5 | VE-Cadherin, Cadherin5 | Involved in ear | |
| | | | development | |
| | STN1 | Stathmin; phosphoprotein | Developmental protein | |
| | | p19; leukemia associated | that controls mammary | |
| | | protein | gland and retina | |
| | | | development. The | |
| | | | expression of this | |
| | | | protein on fetal cells | |
| | | | may be induced by NGF | |
| | | | stimulation | |
| | PRPK | p53 related protein kinase | testes, salivary glands | |
| | RHOB | Transforming protein | Links EGF and PDGF to | |
| | | RhoB | signaling phenomena | |
| | ECP1 | Eosinophil cationic | This protein is known to | |
| | | protein 1 | be expressed in adult | |
| | | | eosinophils and the | |
| | | | pancreas | |
| | RANT | GTP binding nuclear | GTP binding nuclear | |
| | | protein, testis specific | protein, testis specific | |
| | | isoform | isoform | |
| | MTPN | Myotrophin | Synapses in cerebellar | |
| | | | neurons and migration of | |
| | | | granular cells in brain; | |
| | | | cerebellar morphogensis | |
| Q9CQ89 | Q9CQ89; | Divalent cation tolerant | Found in the embryo at | |
| | Q9D1L4 | protein cuta homolog; | day 18 only | |
| | | 0610039D01Rik protein; | | |
| Q9CV89 | Q9CV89 | Adult male tongue cDNA, | Found in the male | |
| | | RIKEN full-length | tongue only | |
| | | enriched library, | | |
| | | clone: 2300010L08, | | |
| | NP25 | Neuronal protein NP25 | Found in adult brain | |
| | PM14 | Pre mRNA branch site | Found in adult breast | |
| | | protein p14 | tissue | |
| ARF6_HUMAN | P26438 | ADP-ribosylation factor 6 | Previously identified in | ARF6 |
| | | | the CD34+ progenitor | |
| | | | cells of the adult bone | |
| | | | marrow | |
| PPOL_MOUSE | Poly | Poly [ADP-ribose] | Required for early fetal | ADPRT; |
| | polymerase | polymerase-1; Poly[ADP- | development and | ADPRT1; |
| | 1P11103; | ribose] synthetase-1; | organogenesis in highly | ADPRP |
| | Q9JLX4; | PARP-1; NAD(+) ADP- | proliferative cells; first | |
| | Q9QVQ3 | ribosyltransferase-1; | expressed at E12.5-liver, | |
| | | msPARP; ADPRT | kidnets, genital ridge, | |
| | | | spinal ganglia; E18.5 | |
| | | | thrymus and NS, trunk, | |
| | | | liver kidney spleen | |
| | | | adrenal glands, stomach, | |
| | | | E14.5 through to adults | |
| | | | it is expressed in the | |
| | | | thymus, testis | |
| ARME_MOUSE | Q9CXI5 | ARMET protein precursor | cDNA found in | ARMET |
| | | | embyonic head | |
| SMD1_HUMAN | P13641 | Small nuclear | Found in placenta, adult | SNRPD1 |
| | | ribonucleoprotein Sm D1; | pancreas, lymphoma | |
| | | Sm-D1; snRNP core | | |
| | | protein D1; Sm-D | | |
| | | autoantigen | | |
| RWD1_MOUSE | Q9CQK7 | RWD domain containing | Found in thymus in adult | RWDD1 |
| | | protein 1; IH1 | | |
| ALFA_MOUSE | P05064 | Fructose-bisphosphate | Found in adult brain and | ALDOA; |
| | | aldolase A; Muscle-type | liver | ALDO1 |
| | | aldolase; Aldolase 1 | | |
| RB27_MOUSE | Q9ERI2 | Ras-related protein Rab- | Found in embryoinic | RAB27A |
| | | 27A | stem cells/mouse. | |
| | | | Restricted to | |
| | | | development | |
| UD11_MOUSE | Q63886 | UDP- | Transferase; | UGT1A1; |
| | | glucuronosyltransferase 1- | Glycosyltransferase; | UGT1 |
| | | 1 precursor, microsomal; | Glycoprotein; | |
| | | UGTBR1; UDPGT; | Transmembrane; Signal; | |
| | | UGT1A1; UGT1.1; | Multigene family; | |
| | | UGT1*1; UGT1-01 | Microsome; Alternative | |
| | | | splicing; similar to Doug | |
| | | | Melton's pancreas library | |
| | | | cDNA, expressed in the | |
| | | | pancreas at E10 | |
| RXRB_MOUSE | P28704; | Retinoic acid receptor | A growth and membrane | RXRB; |
| | P33243 | RXR-beta; MHC class I | receptor for cellular | NR2B2 |
| | | regulatory element | isolations | |
| | | binding protein H-2RIIBP | | |
| NCR2_MOUSE | Q9WU42; | Nuclear receptor | Important for thyroid | NCOR2; |
| | Q9WU43; | corepressor 2; SMRTe; T3 | growth and may interact | SMRT |
| | Q9WUC1 | receptor-associating | with the thyroid | |
| | | factor; Thyroid-, retinoic- | hormone receptor and | |
| | | acid-receptor-associated | retinoic acid receptor | |
| | | corepressor; SMRT; | | |
| | | TRAC; N-CoR2; | | |
| | | Silencing mediator of | | |
| | | retinoic acid and thyroid | | |
| | | hormone receptor | | |
| Q8C9P5 | Q8C9P5 | Bmi1 upstream | | |

### Example 8

### CD180-Deficient Cells as a Target for the Treatment of Diabetes

CD180 (RP150) is a toll-like receptor (TLR) that is critical for the response of B cells to bacterial lipopolysaccharide (LPS). Whole NOD splenocytes from NOD mice greater than 12 weeks of age were analyzed by mass spectrometry for the presence of this protein. Lymphoid cells of B6 mice (normal control) were then separated into non-T-cell and T-cell populations, both of which were similarly analyzed by mass spectrometry. It was found that CD180 protein was detected in the non-T cell fraction of the control mice but not in the NOD mice. As expected, T cells from both NOD mice and B6 mice did not express CD180, as this protein is believed to be restricted to B cells.

It was then found that, in the diabetic mouse, BCG administration kills the subpopulation of B cells that are CD180-deficient. In one experiment, NOD and B6 mice were subjected to BCG treatment via one subcutaneous injection in the footpad. Two days after BCG treatment, the splenocytes were removed and examined for CD180 antigen, at which time both B6 and NOD mice showed equivalent amounts of CD180 antigen in the non-T cell populations. These results were confirmed with analysis by Western gels.

It is known that at least two TLRs expressed on mature B cells (TLR4 and CD180) mediate LPS signaling. The finding that a subpopulation of B cells involved in autoimmunity may be linked to defective CD180 expression and that this subpopulation of autoreactive B cells is eliminated with LPS, or other receptor agonists (such as, for example, those that bind to Toll, TLRs, MD-1, or Ly78) defines a novel way to interfere with autoreactivity in the B cell compartment, therefore identifying a novel therapy for autoimmune diseases (e.g., Type 1 diabetes or lupus) based on the selective killing of disease causing cells.

Agents that can affect the elimination of autoreactive B cells that are deficient in CD180 expression include, small molecule or antibody agonists of TLR1 (such as, for example, triacetylated lipopeptides (LP), phenol-soluble modulin, or OspA LP from B. burgdorferi), small molecule or antibody agonists of TLR2 (such as, for example, LP with TLR1 or TLR6, or HSP60 with TL4), small molecule or antibody agonists of TLR3 (such as, for example, double-stranded RNA), small molecule or antibody agonists of TLR4 (such as, for example, LPS from Gram-negative bacteria, HSP60, mannuronic acid polymers, flavolipins, tecihuronic acids, neumolysin, fimbriae, surfactant protein A, hyaluronan, oligosaccharides, heparin sulfate fragments, fibrinogen peptides, or beta-defensin-2), small molecule or antibody agonists of TLR5 (such as, for example, flagellin), small molecule or antibody agonists of TLR6 (such as, for example, deacetylated LP or phenol-soluble modulin), small molecule or antibody agonists of TLR7 (such as, for example, imidazolquinoline anti-virals), small molecule or antibody agonists of TL8 (such as, for example, imidazolquinoline) or small molecule or antibody agonists of TLR9 (such as, for example, bacterial DNA as CpG motifs).

### Example 9

### Pancreatic, Salivary, and Nerve Tissue Regeneration in NOD Mice

NOD female mice (Taconic Farms, Germantown, N.Y.) as well as male CByF1B6F1/J (CByB6F1) mice (The Jackson Laboratory, Bar Harbor, Me.) were maintained under pathogen-free conditions. Before treatment, NOD females were aged for at least 20 weeks and/or until blood sugar was elevated to levels of 250 mg/dl. Diabetes occurred in 80% of females by 40 weeks of age, with autoimmunity developing preferentially in the female mice.

Splenocytes for treatment of NOD females were obtained from CByB6F1 (H-2KbKdDbDd) male or B6 male mice and injected into these current animals in a live state. The splenocytes (approximately 9×10⁶) were injected into NOD recipients (H-2KdDb) through the tail vein twice a week for 40 days. CFA (Difco, Detroit, Mich.), freshly mixed with an equal volume of physiological saline (50 mL), was also injected into each hind foot pad at the time of islet transplantation or simultaneously with the first splenocyte injection.

Accordingly, seven NOD treatment mice and five NOD control mice were treated with the protocol of CFA plus bi-weekly injections of CD45(−) splenocytes from normal mice. Before the initiation of treatment, all mice were normoglycemic (treatment and untreated groups); All NOD mice, when compared to C57BL/6 control animals, already had a 40-50% reduction in salivary function (the two randomized NOD groups (treated and untreated) had indistinguishable and reduced salivary flow); and all NOD mice suffered from a near 100% hearing loss.

After treatment with CFA and splenocyte injections for 40 days, all 7 of the treated NOD mice were alive, with one of five of the untreated NOD group dying of hyperglycemia. In a comparison of the treated NOD group to the untreated NOD group, salivary flow rates (a test of salivary gland function) in the treated group showed a statistically significant (p=0.009) restoration/stabilization of the salivary flow, suggesting that treatment reduces autoimmunity of the salivary gland. For the treated group, one of the NOD mice demonstrated a 30-40% restoration of hearing. For the untreated group, all the NOD mice remained deaf. Testing of NOD mice rapidly confirmed they were almost 100% deaf by 5 weeks of age.

After 120 days of treatment with CFA and splenocyte injections, one mouse of the treated group died during splenocyte injection. The remaining 6 were normoglycemic (the treated animal that died had a normal blood sugar prior to the death). In contrast, untreated NOD mice were dead from hyperglycemia. For the treated NOD group, salivary flow rates further improved relative to the untreated NOD animals, a but was not yet fully restored to the level found in normal C57BL/6 control mice. For the treated group, the NOD mouse that demonstrated a 30-40% restoration of hearing at 40 days continued to hear at this level. For the untreated group, all the NOD mice remained deaf.

Previously, we demonstrated that diabetes could be reversed in NOD mice (Ryu et al., Journal of Clinical Investigations, 108: 31-33, 2001) and that pancreatic islets could be regenerated without the introduction of exogenous cells. However, the regenerated islets were still susceptible to circumferential insulitis, suggesting that the islets themselves had an intrinsic developmental defect. In the present invention, we describe the isolation from the spleen of an islet precursor CD45(−) pluripotent cell (i.e., AGM cells) in which Hox11 is expressed. As the Hox11 controls islet development, salivary gland development, and multiple forms of neuronal development, including development of the cranial nerves for hearing, the experiments above were conducted to see if CD45(−)/Hox11-expressing splenocytes from normal mice could regenerate neuronal tissue and salivary tissue in NOD mice. The results demonstrate the multi-lineage potential of the CD45(−)/Hox11-expressing AGM cells of the present invention. Although only one of six NOD mice with treatment had hearing function partially restored, the low percentage of hearing loss reversal may be due to the early onset of deafness in NOD mice. Regeneration in middle age may therefore be less efficient.

### Use of AGM Cells for Correction of Cranial Nerve Defects.

The mass spec data of the AGM cells has been shown to express the Hox11 transcription factor. Hox 11 is known to control select cranial nerve development including the growth and development of cranial nerve VIII, the vestibular cochlear nerve. This nerve has a role in hearing.

A strain of mice was identified that had inherent deafness due to a Hox11 lineage defect in the NOD mouse. The NOD mouse is known to have autoimmunity but this can be removed and the underlying hearing defects persists in the NOD-SCID strain of mice. Neuro-anatomy of these mice reveals that this defect caused a specific traceable lesion in the spiral limbus of the mice, apparent as early as 5 weeks of age. Furthermore live mice can be tested for auditory function and auditory high frequency loss is almost uniformly present by 5-8 weeks of age in either NOD or NOD-SCID mice. This auditory loss is not due to infection of the mice with middle ear infections. In a protocol identical to our past published protocols (Kodama et al, Science 2003), we treated NOD mice with CD45−, CD45+ and whole splenocytes for the 40 day protocol combined with CFA. CFA was chosen as an agent that induces high levels of endogenous TNF. TNF is not only needed in these mice to selective kill the atuoreactive cells but TNF is thought to stimulate the animals to promote regeneration. This is reinforced by the mass spectroscopy data that now reveals that the introduced AGM cells express TNF receptor I (TNFR1) while non-AGM cells, i.e. CD45+ cells, lack this receptor. Therefore, boosting the levels of TNF by any number of means appears to promote cellular regeneration.

End stage hearing defects are not known to be reversible. All mice used in this study were end-stage with respect to hearing loss prior to initiation of the intervention, i.e. greater than 90% hearing loss at all frequencies. Hearing was monitored at two time points after therapy, i.e., 40 days and 80 days in the treatment groups composed of 5 mice each, i.e., CD45− treated, CD45+ treated, whole splenocyte treated, and untreated. At both 40 and 80 days after therapy initiation, hearing was still absent in the NOD groups receiving CD45+ cells and in the untreated mice. In contrast, the NOD mice receiving CD45− cells or whole splenocytes had partial or full recovery of their hearing at both monitoring time points. In the CD45− treatment group, two cohorts had greater than 60% recovery of hearing; the other three cohorts had 30-50% recovery of hearing function. The recovery of hearing persisted through the next 40-day monitoring period.

### Use of AGM Cells to Correct Salivary Disease.

The mass spectroscopy data of the AGM cells expressing Hox11 implies that these cells are capable of controlling salivary gland development. Thus, the salivary gland is one of the endocrine glands capable of benefiting from therapy involving the administration of AGM cells to the patient.

As outlined above, the non-obese diabetic (NOD) mice are a model for deafness. These mice can also be used as a model for Sjögren's disease, an autoimmune disease of the salivary glands. We have devised an intervention with complete Freund's adjuvant (CFA) combined with matched splenocytes that permanently reverses established end stage diabetes and these cells have been shown to differentiate into insulin secreting beta cells. We now replicate these findings and focus on the impact of this therapy on advanced Sjögren's disease in the autoimmune prone NOD mice.

Severe end-stage mice with Sjögren's disease were monitored for salivary flow. If salivary flow was greater than 50% suppressed, then the NOD mice were treated with the above treatment. As is also reported in the literature, Sjögren's disease occurs prior to the other autoimmune diseases of these mice, i.e. diabetes. To simplify these experiments, all NOD recipient mice were female and still normoglycemic at the start of therapy at 10-14 weeks of age. The recipient mice were treated with male donor spleen cells to allow for the later examination of tissue for chimerism using fluorescence in situ hybridization (FISH). This method detects the Y chromosome of the male donor cells and allows rapid and facile detection of the donor AGM cells. Of the five NOD mice treated with CFA and spleen cells, salivary flow diminished during the first 40 days of the treatment period. Over the second 40 day treatment period, the treated mice slowly exhibited continuous recovery of salivary flow. At the 80-day end point, the treated mice exhibited normal salivary flow. All untreated NOD mice died during the 80-day treatment interval due to the onset of diabetic hyperglycemia. Thus, treatment with CPA and spleen cells reversed both diabetic and Sjögren's autoimmunity with remarkable success.

To confirm that recovery of the treated mice resulted from engraftment of the administered AGM cells, the successfully treated animals were killed and one salivary gland was evaluated both for histology removal of salivary infiltrates and evidence of the persistence of Y positive, non-lymphoid cells in the salivary glands (from the male donor). For these cohorts of successfully treated mice, a histologic difference in salivary gland autoimmune infiltrates could not be readily observed. But what was observed was the clear presence of Y positive cells of non-lymphoid origins within the salivary tissues of the treated mice. These Y positive cells made up about 10-15% of the cells in the salivary gland and were not found in other sites in the animal, such as the heart, lung, and kidney. As expected, the animals did have well granulated islets from the therapy and the islets contain insulin positive cells with the Y chromosome.

These experiments demonstrate that AGM cells of the spleen can differentiate into both pancreatic islets and cells of the salivary gland. These experiments also establish that AGM cells are capable of reversing end-stage deafness in some cohorts of mice.

### Example 10

### Treatment of Patients with Compositions Enriched in CD45(−)pluripotent Cells that Express Hox11

While the therapies described herein are likely to be effective in treating pre-diabetics, i.e., patients diagnosed as progressing to type I diabetes, but who are not yet hyperglycemic, we note that the methods of the inventions also may be used to treat a mammal, for example, a human with type I diabetes or any other autoimmune disease. The ability to treat patients who already have hyperglycemia and therefore have significant or total islet destruction is a significant advantage of the current therapy.

In general, before treating a patient with a composition that includes AGM cells of the invention, one may optionally obtain blood from the patient to determine that the patient has two disease phenotypes. The first disease phenotype is an increase in the number of circulating CD45RA positive cells in the blood (also defined as alterations in the number of cells positive for CD95, CD62L, or other markers of naive or unstimulated cells). CD45, CD95, and CD62L are all cell surface antigens that can be monitored by flow cytometry and compared to age matched controls. We expect to see an abundance of these naïve or unstimulated cells in the peripheral blood of subjects with diabetes or any other autoimmune disease. The second phenotype is the presence of a subpopulation of lymphocytes with augmented sensitivity to cell death through apoptosis or necrosis. For example, subpopulations of cells may have augmented sensitivity to cell death caused by TNF-alpha, TCR receptor cross-linking agents, T-cell specific antibodies (e.g., αTCR or αCD3), or nonspecific stimulation with BCG. We may assay for the presence of such cells by isolating lymphocytes from these patients, treating them in vitro with TNF-alpha, and showing that the lymphocytes contain a subpopulation that undergoes apoptosis or necrosis when exposed to TNF-alpha, other cytokines, chemical reagents, or antibodies to select surface proteins. Desirably, control donor lymphocytes do not exhibit sensitivity to these agents. This phenotype is a result of lymphoid cells predominantly of pathogenic origin that have altered intracellular signaling pathways, alterations which result in a heightened death sensitivity. Elimination or conversion of all cells with this phenotype is desirable for the permanent reversal of autoimmunity. The penetrance of these defects is likely to be relatively high in diabetic or other autoimmune patients, with the first phenotype likely having a penetrance of over 95%, and the second phenotype likely having a penetrance of over 50% in type I diabetics.

Accordingly, before beginning to treat a subject with type I diabetes or any other autoimmune condition, we may determine from blood analysis alone whether the subject has either or both of these two phenotypes and, therefore, is amenable to therapy. To treat the first phenotype (i.e., an increase in the number of circulating CD45RA positive cells) tolerance to MHC class I and self-peptide may have to be re-established. We conclude from our results that the lack of functional MHC class I and self-peptide complexes causes the overabundance of naïve T-cells in the periphery or at least results in one of the phenotypes that causes this. So for treating this phenotype, we can administer blood or bone marrow that is a semi-allogeneic or fully-allogeneic match to the MHC class I and self-peptide complex. Furthermore, the blood or bone marrow derived cells, or even fibroblasts that have been immortalized, desirably may have normal MHC class I and self-peptide complex presentation; in other words, they should not come from diseased patients. Those phenotypes are easily monitored prior to treatment to determine the suitability of the donor cells in this therapy. For example, conformationally specific MHC class I and self-peptide antibodies may be used to show that the complexes are properly filled. In addition, we know that, in this aspect of the treatment, an increased number of matches to the HLA class I alleles of the host results in an increase in the duration of the reversal of the disease. Desirably, at least two, and desirably all four HLA class I alleles (e.g., the HLA A and HLA B alleles) from the donor cells are matched. Accordingly, these donor cells may be perfectly matched or they may be semi-allogeneic (i.e., with only partial matches on individual cells).

Treatment may involve intravenous biweekly infusions of, e.g., 1×10⁷ cells of any given donor of any given class I haplotype. It is desirable for the administered cells to be freshly isolated and not processed with preservatives or frozen. Cells that may be used in the methods of the invention may be obtained, for example, from a bloodbank. In addition, semi-allogeneic cells may be obtained from a close relative of the patient, such as a parent or a sibling. Furthermore, it would be advantageous to have the red blood cells eliminated from the preparations to decrease the volume of blood and lymphocytes administered. Alternatively, CD45(−) pluripotent cells (e.g., splenocytes, or those derived from cord blood or embryonic stem cells) can be transfected with a gene for Hox 11, preferably a human gene, or induced to express Hox 11 and the resulting cells used for treatment.

As an alternative to administering MHC class I and peptide, another agent that inactivates or kills naive T-cells can be administered. Exemplary agents include antibodies that bind and inactivate the T-cell receptor on naive T-cells or by binding and triggering the selective death of only pathologic cells. In some embodiments, the antibodies inhibit the activity of naive T-cells by at least 2, 5, 10, or 15-fold more than they inhibit the activity of memory T-cells.

Simultaneously with the administration of donor cells, it is also desirable to induce endogenous TNF-alpha production either through stimulation with Bacillus Clamette-Guerin (BCG) or other immune adjuvants such as CFA, or by the direct administration of TNF-alpha. For example, one may administer BCG at least biweekly or, desirably, three times a week. Again, one skilled in the art can determine individually the dosing of the cells and TNF-alpha or BCG by analyzing a blood sample twice a week for evidence of the elimination of the phenotype of the pathogenic cell. For instance, to determine the correct dose of donor MHC class I expressing cells, we may look for the elimination of the abundant naïve cells in the peripheral blood and to determine the correct dose of TNF-alpha or BCG, we may look for the elimination of TNF-alpha in vitro sensitivity.

With regard to the second aspect of the therapy, TNF-alpha, BCG, or another nonspecific form of immune stimulation may promote the induction of endogenous TNF-alpha. For example, TNF-alpha may be administered intramuscularly, intravesicularly, or intravenously. Moreover, recombinant human TNF-alpha or new drugs such as a TNF receptor 2 agonist may be used. Such compounds have two effects, one is the elimination of apoptosis or death sensitive cells in the periphery which can be monitored, and the other is the promotion of endogenous beta cell regeneration, as well as possibly differentiation from the new donor blood. Exemplary doses of TNF-alpha that may be administered to a patient are approximately 40 μg/m² or 200 μg/m². Other exemplary doses include doses between 2×10⁶ and 5×10⁶ mg daily for two doses in one week. Patients with an autoimmune disease may tolerate higher doses of TNF-alpha and/or may require lower doses for treatment. As an alternative to TNF-alpha, tolerance can be gained by cross-linking the TCR or by nonspecific vaccination through the same pathway (e.g., BCG vaccination). As an alternative to administering an inducer of T-lymphopenia (e.g., TNF-alpha) directly to a patient, the inducer of T-lymphopenia can be administered to blood obtained from the patient and the treated blood can be re-administered to the patient. For inducers of T-lymphopenia with a short half-life (e.g., TNF-alpha) little, if any, functional compound remains in the blood that is re-introduced into the patient. Thus, this method should decrease the incidence or severity of any potential adverse, side-effects of the compound.

Any combination therapy described herein, e.g. a therapy which uses MHC class I expressing cells and TNF-alpha induction, may be administered until the disease is successfully treated. For example, this therapy may be continued for approximately 40 days; however, this time-period may readily be adjusted based on the observed phenotypes. Additionally, the dose of TNF-alpha can be adjusted based on the percentage of cells in blood samples from the patient that have increased sensitivity to TNF-alpha, indicating the amount of remaining autoimmune cells. In addition, in treating type I diabetes, it may be desirable that the patient maintains as close to normoglycemia as possible. The murine data have demonstrated that marked fluctuation in blood sugars hamper the normal regenerative potential of the pancreas. Therefore, these patients may be placed on an insulin pump for not only the exemplary 40 days of disease reversing therapy, but also for a 120 day period to optimize the regenerative process. The pancreas of long-term diabetics (i.e., ones having diabetes for more than 15 years) may have the regenerative potential of the pancreas diminished to such a degree that the precursor cells are no longer present. In these patients, the therapy may be identical except for the length of the treatment. For instance, the donor blood or bone marrow cells have to be alive for these cells to convert to the correct tissue type, such as into beta cells of the pancreas.

As is mentioned above, some embodiments of the invention employ CD45(−) pluripotent cells (e.g., from the bone marrow, the spleen, or the peripheral blood, preferably from the spleen) that express Hox 11, isolated from self or a normal donor. Typically, this cell expresses, to a detectable degree, CD90⁺, CD44⁺, or CD29⁺, but does not express appreciable amounts of CD34. This normal donor cell is administered to a person, preferably intravenously or intraperitoneally, to allow for rapid transport to the site of inflammation, injury, or disease. Desirably, this cell is administered to a person with active autoimmunity. Alternatively, the cell may be administered to a person without autoimmunity or to a person with quiescent autoimmunity. The absence of active autoimmunity in a person (host) may require pretreatment of the host to initiate an inflammatory response or injury at the regenerative site. In addition, pretreatment of the donor cell may also be required. The host may be treated with TNF-α, IFN-γ, IL-2, VEGF, FGF, or IGF-1 to prepare the blood vessel endothelium for optimal interactions with the mobilized Hox 11-expressing cell. Additionally, the pathway of VEGF-stimulated expression on endothelial cells can be enhanced with a selective inhibitor of PI-3′-kinase. Alternatively, the host can be pretreated with platelet-derived growth factor derived from mural cells (e.g., from the neural crest or epicardium) for optimal interactions with the mobilized mesodermal cell. Additionally, the mesodermal cell can be pretreated to optimize adherence to the endothelium. This type of therapy is envisioned to be beneficial for the regeneration of diverse organs or organelles, including islets of Langerhans, liver, pancreas, spleen, bone, and other organs and glands of the body.

### Other Embodiments

From the foregoing description, it will be apparent that variations and modifications may be made to the invention described herein to adapt it to various usages and conditions. Such embodiments are also within the scope of the following claims.

All publications mentioned in this specification are herein incorporated by reference to the same extent as if each independent publication, patent, or patent application was specifically and individually indicated to be incorporated by reference.

What is claimed is:

1. A cell-containing composition, wherein at least 10% of the cells of said composition comprise aorta gonad mesoderm (AGM) cells.

2. The composition of claim 1, wherein at least 95% of the cells of said composition comprise AGM cells.

3. The composition of claim 1, wherein said AGM cells express Hox11.

4. The composition of claim 1, wherein said AGM cells do not express CD45.

5. The composition of claim 1, wherein said AGM cells express one or more cell markers selected from the group consisting of: retinoic acid receptor, estrogen receptor, EGF receptor, CD49b, VLA2, CD41, LFA-1, ITB2, CD29, NTC3 receptor, plasminogen receptor, transferrin receptor, TGF receptor, TNF receptor, PDGF receptor, thyroid growth hormone receptor, and integrin beta 5.

6. The composition of claim 1, wherein said AGM cells are obtained from the peripheral blood or tissue of a mammal.

7. The composition of claim 1, wherein said AGM cells are obtained from spleen, tonsils, adenoids, thymus, peripheral blood, or cord blood.

8. The composition of claim 7, wherein said AGM cells are obtained from the capsule or pulp of the spleen.

9. The composition of claim 1, wherein said AGM cells are capable of differentiating into pancreatic cells, spleen cells, salivary gland cells, liver cells, kidney cells, nerve cells, or bone cells.

10. The composition of claim 1, wherein said AGM cells are capable of regenerating an organ or a tissue from an organ, wherein the organ or tissue is selected from pancreas, salivary gland, pituitary gland, kidney, heart, olfactory gland, ear, bone, liver, brain, peripheral nervous system, central nervous system, spinal cord, mammary gland, or testes.

11. The composition of claim 1, wherein said AGM cells are human cells.

12. The composition of claim 1, wherein said composition comprises a pharmaceutically acceptable carrier.

13. The composition of claim 1, wherein said composition further comprises an agent that selectively inhibits, removes, or kills a cell population that interferes or prevents the trafficking of, differentiation of, or growth of said pluripotent cells.

14. The composition of claim 13, wherein said cell population comprises T-lymphocytes.

15. The composition of claim 13, wherein said agent is BCG, lipopolysaccharide, (LPS), a triacetylated lipopeptide, phenol-soluble modulin, OspA lipopeptide from B. burgdorferi, a triacetylated lipopeptide with TLR1 or TLR6, HSP60 with TL4, HSP60, a mannuronic acid polymer, a flavolipin, a tecihuronic acid, neumolysin, fimbriae, surfactant protein A, hyaluronan, heparin sulfate or a heparin sulfate fragment, a fibrinogen peptide, beta-defensin-2, flagellin, imidazolquinoline, TNF-alpha, a TNF-alpha agonist, or a TNF-alpha inducing substance.

16. The composition of claim 15, wherein said agent is TNF-alpha.

17. The composition of claim 15, wherein said agent is a TNF-alpha agonist or a TNF-alpha inducing substance.

18. The composition of claim 17, wherein said TNF-alpha agonist or TNF-alpha inducing substance is Complete Freund's Adjuvant (CFA), ISS-ODN, microbial cell wall components with LPS-like activity, cholera particles, E. coli heat labile enterotoxin, E. coli heat labile enterotoxin complexed with lecithin vesicles, ISCOMS-immune stimulating complexes, polyethylene glycol, poly(N-2-(hydroxypropyl)methacrylamide), synthetic oligonucleotides containing CpG or CpA motifs, monophosphoryl lipid A, Bacillus Clamette-Guerin, γ-interferon, Tissue Plasminogen Activator, LPS, Interleukin-1, Interleukin-2, UV light, a lymphotoxin, cachectin, a TNFR-1 agonist, a TNFR-2 agonist, an intracellular mediator of the TNF-alpha signaling pathway, a NFκB inducing substance, IRF-1, STAT1, a lymphokine, or the combination of TNF-alpha and an anti-TNFR-1 antibody.

19. The composition of claim 18, wherein said TNF-alpha agonist or TNF-alpha inducing substance is Complete Freund's Adjuvant, Bacillus Clamette-Guerin, or γ-interferon.

20. The composition of claim 1, wherein said composition further comprises a cytokine, a chemokine, or a growth factor.

21. The composition of claim 20, wherein said cytokine, chemokine, or growth factor is selected from the group consisting of: epidermal growth factor (EGF), platelet-derived growth factor (PDGF), fibroblast growth factors (FGFs), transforming growth factor-beta (TGF-β), transforming growth factor-alpha (TGF-α), vascular endothelial growth factor (VEGF), erythropoietin (Epo), insulin-like growth factor-I (IGF-I), insulin-like growth factor-II (IGF-II), interleukins, tumor necrosis factor-alpha (TNF-α), tumor necrosis factor-beta (TNF-β), interferon-gamma (INF-γ), stromal cell-derived factor-1 (SDF-1), and a colony stimulating factors (CSF).

22. A method for treating or preventing a disease or disorder or for reconstituting the immune system of a mammal, comprising administering the composition of claim 1.

23. The method of claim 22, wherein said disease or disorder is an autoimmune disease or disorder.

24. The method of claim 23, wherein said autoimmune disease or disorder is selected from alopecia areata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, systemic lupus erythmatosous, ulcerative colitis, psoriatic arthritis, essential mixed cryoglobulinemia, fibromyalgia-fibromyositis, Graves' disease, Guillain-Barré, Hashimoto's thyroiditis, hypothyroidism, idiopathic pulmonary fibrosis, idiopathic thrombocytopenia purpura (ITP), IgA nephropathy, insulin dependent diabetes, juvenile arthritis, lichen planus, lupus, Ménière's disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, Stiff-Man syndrome, Takayasu arteritis, temporal arteritis/giant cell arteritis, ulcerative colitis, uveitis, vasculitis, vitiligo, and Wegener's granulomatosis.

25. The method of claim 24, wherein said autoimmune disease or disorder is insulin dependent diabetes.

26. The method of claim 24, wherein said autoimmune disease or disorder is Sjögren's syndrome.

27. The method of claim 22, wherein said mammal is a human.

28. A method for increasing, maintaining, or replenishing the number of functional cells of a predetermined type in an organ or tissue of a mammal, wherein said organ or tissue is injured, damaged, or deficient in said functional cells, said method comprising administering to said mammal the composition of claim 1.

29. The method of claim 28, wherein said organ or tissue is, or is part of, the pancreas, salivary gland, pituitary gland, kidney, heart, olfactory gland, ear, bone, liver, brain, peripheral nervous system, central nervous system, spinal cord, mammary gland, or testes.

30. The method of claim 29, wherein said organ or tissue is, or is part of, the pancreas.

31. The method of claim 28, wherein said mammal has an autoimmune disease or disorder.

32. The method of claim 31, wherein said autoimmune disease or disorder is selected from alopecia areata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, discoid lupus, systemic lupus erythmatosous, ulcerative colitis, psoriatic arthritis, essential mixed cryoglobulinemia, fibromyalgia-fibromyositis, Graves' disease, Guillain-Barré, Hashimoto's thyroiditis, hypothyroidism, idiopathic pulmonary fibrosis, idiopathic thrombocytopenia purpura (ITP), IgA nephropathy, insulin dependent diabetes, juvenile arthritis, lichen planus, lupus, Ménière's disease, mixed connective tissue disease, multiple sclerosis, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, Stiff-Man syndrome, Takayasu arteritis, temporal arteritis/giant cell arteritis, ulcerative colitis, uveitis, vasculitis, vitiligo, and Wegener's granulomatosis.

33. The method of claim 32, wherein said autoimmune disease or disorder is insulin dependent diabetes.

34. The method of claim 32, wherein said autoimmune disease or disorder is Sjögren's syndrome.

35. The method of claim 32, wherein said autoimmune disease or disorder is diabetes.

36. The method of claim 32, wherein said autoimmune disease or disorder is immunologically-mediated glomerulonephritis.

37. The method of claim 32, wherein said autoimmune disease or disorder is chronic hepatitis, primary biliary cirrhosis, or primary sclerosing cholangitis.

38. The method of claim 28, wherein said mammal is a human.

39. A method for preparing a cell-containing composition comprising aorta gonad mesoderm (AGM) cells comprising:
a. providing tissue from a mammal, wherein said tissue is obtained from spleen, tonsil, adenoid, thymus, peripheral blood, or cord blood of said mammal;
b. separating from said tissue a first cell population which predominantly expresses CD45 antigen and a second cell population which predominantly does not express CD45 antigen; and
c. selecting said second cell population;
wherein at least 10% of the cells of said second cell population comprise said AGM cells.

40. The method of claim 39, wherein at least 90% of the cells of said second cell population composition comprise said AGM cells.

41. The method of claim 39, further comprising:
d. further separating said AGM cells from non-AGM cells using one or more cell surface markers expressed by said AGM cells selected from the group consisting of: retinoic acid receptor, estrogen receptor, EGF receptor, CD49b, VLA2, CD41, LFA-1, ITB2, CD29, NTC3 receptor, plasminogen receptor, transferrin receptor, TGF receptor, TNF receptor, PDGF receptor, thyroid growth hormone receptor, and integrin beta 5.

42. The method of claim 39, wherein said tissue is obtained from the capsule or pulp of the spleen.

43. The method of claim 39, wherein said AGM cells express Hox11.

44. The method of claim 39, wherein said mammal is a human.

45. Use of a cell-containing composition comprising at least 10% AGM cells in the manufacture of a medicament for treating or preventing a disease or disorder or for reconstituting the immune system of a mammal.

46. Use of a cell-containing composition comprising at least 10% AGM cells in the manufacture of a medicament for increasing, maintaining, or replenishing the number of functional cells of a predetermined type in an organ or tissue of a mammal, wherein said organ or tissue is injured, damaged, or deficient in said functional cells.
